(19) United States
(12) Patent Application Publication
(10) Pub. No.: US 2012/0101098 A1
(43) Pub. Date: Apr. 26, 2012

(54) 3,28-DISUBSTITUTED BETULINIC ACID DERIVATIVES AS ANTI-HIV AGENTS

(76) Inventors: Kuo-Hsiung Lee, Chapel Hill, NC (US); Keduo Qian, Carrboro, NC (US); Donglei Yu, Bethesda, MD (US); Chin-Ho Chen, Chapel Hill, NC (US); Li Huang, Chapel Hill, NC (US); Ibrahim Danlami Bori, Durham, NC (US)

(21) Appl. No.: 13/320,642

(22) PCT Filed: May 10, 2010
(86) PCT No.: PCT/US10/34193

§ 371 Date: Dec. 16, 2011

Related U.S. Application Data
(60) Provisional application No. 61/178,516, filed on May 15, 2009.

(51) Int. Cl. A61K 31/5377; C07D 211/34; C07D 413/06; C07D 413/12; A61P 31/18; A61K 31/194; A61K 31/445; A61K 31/215; A61K 31/25; A61P 31/14; C07C 235/82; C07C 69/753

(52) U.S. Cl. 514/235.5; 562/403; 546/195; 544/130; 560/6; 514/563; 514/319; 514/237.2; 514/510

(57) ABSTRACT

Compounds according to Formula (I) are described along with compositions containing the same and methods of use thereof for the treatment of viral infections.

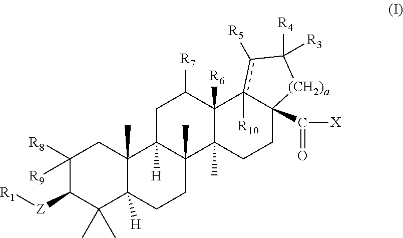

### RELATED APPLICATION DATA

This application claims the benefit of U.S. Provisional Patent Application Ser. No. 61/178,516, filed May 15, 2009, the disclosure of which is incorporated herein by reference in its entirety.

### GOVERNMENT FUNDING

This invention was made with United States Government support under grant number AI 077417 from the National Institute of Allergy and Infectious Diseases. The United States government has certain rights to this invention.

### FIELD OF THE INVENTION

The present invention concerns compounds, compositions and methods useful for the treatment of retroviral infections in human or animal subjects in need thereof.

### BACKGROUND OF THE INVENTION

As the world enters the third decade of the AIDS epidemic, this pandemic has rapidly grown into the fourth leading cause of mortality globally.¹ Introduction of highly active antiretroviral therapy (HAART), which employs a combination of nucleoside/nucleotide reverse transcriptase inhibitors (NRTIs), non-nucleoside reverse transcriptase inhibitors (NNRTIs), and/or protease inhibitors (PIs), has significantly improved the treatment of HIV/AIDS.²⁻⁵ However, the virus is suppressed rather than eradicated by HAART.⁶⁻⁸ On HAART regimens, multiple drug therapies can lead to increased adverse effects and toxicities due to long-term use and drug-drug interactions.^{9,10} Moreover, it inevitably leads to the emergence of multi-drug-resistant viral strains.¹¹ In fact, a significant proportion of newly infected individuals harbor HIV-1 isolates that are resistant to at least one ART.^{12,13} Therefore, novel potent antiretroviral agents are needed, with different targets than currently approved drugs and preferably with simplified treatment regimens (fewer pills and less-frequent administration).

Triterpenes, such as betulinic acid (BA, 1), represent a promising class of anti-HIV agents with novel mechanisms. Two types of BA derivatives have exhibited potent anti-HIV profiles. C-3 esterification of BA led to the discovery of bevirimat (DSB, PA-457, 2), which is a HIV-1 maturation inhibitor (MI) that blocks cleavage of p25 (CA-SP1) to functional p24 (CA), resulting in the production of noninfectious HIV-1 particles.¹⁴⁻¹⁶ Bevirimat (2) is currently in Phase IIb clinical trials launched by Panacos Pharmaceuticals, Inc.^{17,18} On the other hand, the C-28 side chain was proven to be a necessary pharmacophore for anti-HIV entry activity, as seen with the equipotent diastereomers RPR103611 (3a) and IC9564 (3b).¹⁹⁻²² Mechanism of action studies have revealed that C-28 modified BA derivatives function at a post-binding, envelope-dependent step involved in fusion of the virus to the cell membrane.²³ Recent studies further suggested that 3b may also function by targeting the V3 loop of gp120, a domain involved in chemokine receptor binding.²⁴ Although 3a showed potent antiviral activity in vitro, the clinical development of 3a by Rhone-Poulenc (now Sanofi-Aventis) was stopped due to poor “pharmacodynamic properties”.²⁵

### SUMMARY OF THE INVENTION

A first aspect of the invention is a compound according to Formula (I):

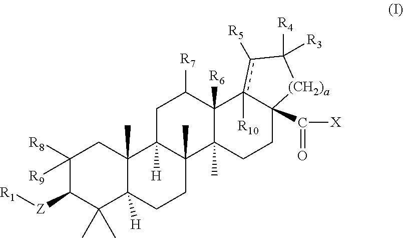

wherein:

a is 1 or 2;

Z is O, S, NH, or N-alkyl;

R₁ is a hydrogen, acyl carboxylic acid, C₂ to C₂₀ substituted or unsubstituted carboxyacyl, or a substituent of the formula:

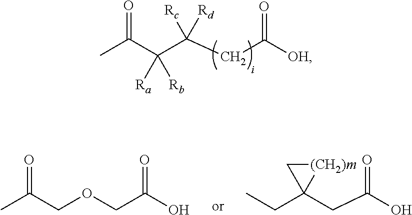

wherein Rₐ, R_{b}, R_{c} and R_{d} are the same or different and are each independently selected from the group consisting of hydrogen and lower alkyl, i is an integer from 0 to 3, and m is an integer from 1 to 4;

X is polyalkylene oxide, heteroalkylene, or —NR₂ₐR_{2b}, wherein R₂ₐ is H, loweralkyl, heteroalkylene, or polyalkylene oxide and R_{2b} is H, heteroalkylene, polyalkylene oxide, or a substituent of the formula:

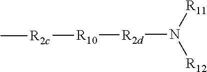

where R_{2c} is C2 to C10 saturated or unsaturated alkylene, R_{2d} is present or absent and when present is C1 to C5 saturated or unsaturated alkylene, R₁₀ is CONH, NHCO, NH, SH, or O, and R₁₁ and R₁₂ are each H, loweralkyl, heteroalkyl, carboxy, amino acid, or a peptide, or R₁₁ and R₁₂ together form with the N to which they are joined cycloalkyl or heterocycloalkyl;

or R₂ₐ and R_{2b} together are C3 to C5 alkylene, which alkylene is substituted or unsubstituted;

R₃ and R₄ are either H or lower alkyl (e.g., methyl);

R₅ is H, lower alkyl, or —CRᵢRᵢᵢRᵢᵢᵢ, where:
Rᵢ is a methyl radical or forms with Rᵢᵢ a methylene radical or an oxo radical;
Rᵢᵢ is a hydrogen atom or forms with Rᵢ or Rᵢᵢᵢ a methylene radical or an oxo radical; and
Rᵢᵢᵢ is a hydroxyl, methyl, hydroxymethyl, —CH₂OR′ᵢᵢᵢ, —CH₂SR′ᵢᵢᵢ, or —CH₂NHR′ᵢᵢᵢ, which R′ᵢᵢᵢ is alkyl, hydroxyalkyl, dihydroxyalkyl, acetamidoalkyl, acetyl, heteroalkylene, or polyalkylene oxide; or Rᵢᵢᵢ is an amino radical substituted with hydroxyalkyl, carboxyhydroxyalkyl, or dialkylamino, the alkyl parts of which can form, with the nitrogen atom to which they are joined, a 5- or 6-membered heterocycle optionally containing 1 or 2 additional hetero atoms selected from the group consisting of O, S, NH, and N-alkyl;
   or R₅ is a bond to an immediately adjacent carbon atom (thus forming a double bond in the ring between immediately adjacent carbon atoms);

R₆ and R₇ are either H or form a bond with one another (thus forming a double bond between their immediately adjacent carbon atoms);

R₈ and R₉ are either hydrogen or together form an oxo radical;

R₁₀ is either H or a bond with an immediately adjacent carbon atom (thus forming a double bond in the ring between immediately adjacent carbon atoms); and

the dashed line in Formula (I) is an optional double bond;

or a stereoisomer, enantiomer, tautomer thereof or mixtures thereof;
or a pharmaceutically acceptable salt or prodrug thereof.

Scheme A herein shows the structures of Betulinic acid, Bevirimat, and representative compounds of the present invention.

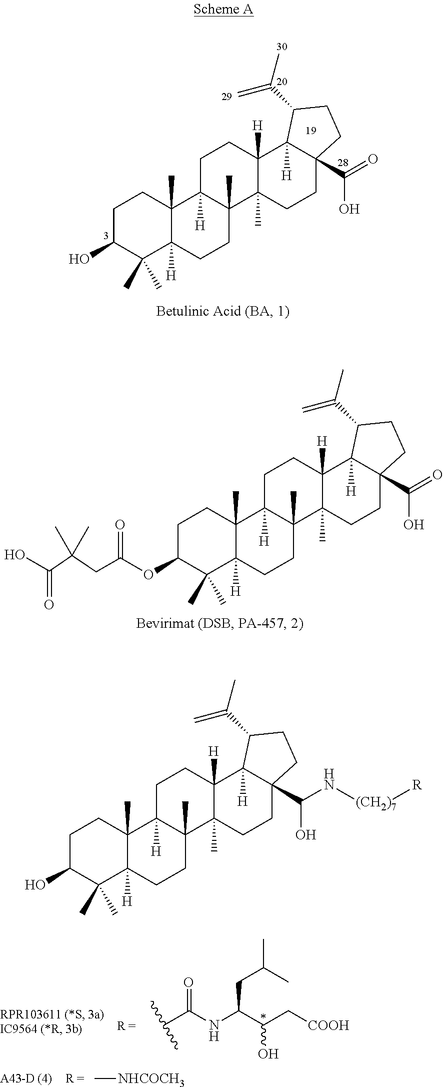

A further aspect of the present invention is a composition comprising a compound of Formula (I) (an active compound) in a pharmaceutically acceptable carrier (such as an aqueous carrier).

A further aspect of the present invention is a composition comprising a compound of Formula (I) (an active compound) in a pharmaceutically acceptable carrier (such as an aqueous carrier) and one or more additional antiviral agent such as an HIV entry inhibitor.

A further aspect of the present invention is directed to methods for treating a viral infection, particularly a retroviral infection (e.g., HIV-1 infection) in cells or tissue of an animal, in an animal subject or human, comprising administering an effective retroviral inhibiting amount of a compound of Formula (I). The examples of HIV infection includes, but not limit to, DSB-resistant HIV-1 infection and RPR103611-resistant HIV-1 infection, etc.

The present invention is explained in greater detail in the specification set forth below.

### DETAILED DESCRIPTION OF THE PREFERRED EMBODIMENTS

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs. All publications, patent applications, patents and other references mentioned herein are incorporated by reference in their entirety.

“Moiety” and “group” are used interchangeably herein to refer to a portion of a molecule, typically having a particular functional or structural feature, e.g. a linking group (a portion of a molecule connecting two other portions of the molecule).

“Substituted” as used herein to describe chemical structures, groups, or moieties, refers to the structure, group, or moiety comprising one or more substituents. As used herein, in cases in which a first group is “substituted with” a second group, the second group is attached to the first group whereby a moiety of the first group (typically a hydrogen) is replaced by the second group. The substituted group may contain one or more substituents that may be the same or different.

“Substituent” as used herein references a group that replaces another group in a chemical structure. Typical substituents include nonhydrogen atoms (e.g. halogens), functional groups (such as, but not limited to amino, sulfhydryl, carbonyl, hydroxyl, alkoxy, carboxyl, silyl, silyloxy, phosphate and the like), hydrocarbyl groups, and hydrocarbyl groups substituted with one or more heteroatoms. Exemplary substituents include, but are not limited to, alkyl, lower alkyl, haloalkyl, alkenyl, alkynyl, cycloalkyl, cycloalkylalkyl, heterocyclo, heterocycloalkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, boronyl, and modified lower alkyl.

“Alkyl” as used herein alone or as part of another group, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. The term “alkyl” or “loweralkyl” is intended to include both substituted and unsubstituted alkyl or loweralkyl unless otherwise indicated and these groups may be substituted with groups selected from polyalkylene oxides (such as PEG), halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Alkenyl” as used herein alone or as part of another group, refers to a straight or branched chain hydrocarbon containing from 1 to 10 carbon atoms (or in loweralkenyl 1 to 4 carbon atoms) which include 1 to 4 double bonds in the normal chain. Representative examples of alkenyl include, but are not limited to, methylene (═CH₂), vinyl, 2-propenyl, 3-butenyl, 2-butenyl, 4-pentenyl, 3-pentenyl, 2-hexenyl, 3-hexenyl, 2,4-heptadiene, and the like. The term “alkenyl” or “loweralkenyl” is intended to include both substituted and unsubstituted alkenyl or loweralkenyl unless otherwise indicated and these groups may be substituted with groups such as those described in connection with alkyl and loweralkyl above.

“Alkynyl” as used herein alone or as part of another group, refers to a straight or branched chain hydrocarbon containing from 1 to 10 carbon atoms (or in loweralkynyl 1 to 4 carbon atoms) which include 1 triple bond in the normal chain. Representative examples of alkynyl include, but are not limited to, 2-propynyl, 3-butynyl, 2-butynyl, 4-pentynyl, 3-pentynyl, and the like. The term “alkynyl” or “loweralkynyl” is intended to include both substituted and unsubstituted alkynyl or loweralkynyl unless otherwise indicated and these groups may be substituted with the same groups as set forth in connection with alkyl and loweralkyl above.

“Cycloalkyl” as used herein alone or as part of another group, refers to a saturated or partially unsaturated cyclic hydrocarbon group containing from 3, 4 or 5 to 6, 7 or 8 carbons (which carbons may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or loweralkyl. The term “cycloalkyl” is generic and intended to include heterocyclic groups as discussed below unless specified otherwise.

“Cycloalkylalkyl” as used herein refers to a cycloalkyl group, as defined herein, that is substituted with an alkyl group, as defined herein. Either the alkyl group or the cycloalkyl group may be attached to the parent molecular moiety and either group may be further substituted as defined herein.

“Cycloalkylalkenyl” as used herein refers to a cycloalkyl group, as defined herein, that is substituted with an alkenyl group, as defined herein. Either the alkenyl group or the cycloalkyl group may be attached to the parent molecular moiety and either group may be further substituted, as defined herein.

“Cycloalkylalkynyl” as used herein refers to a cycloalkyl group, as defined herein, that is substituted with an alkynyl group, as defined herein. Either the alkynyl group or the cycloalkyl group may be attached to the parent molecular moiety and either group may be further substituted, as defined herein.

“Heterocyclic group” or “heterocyclo” as used herein alone or as part of another group, refers to an aliphatic (e.g., fully or partially saturated heterocyclo) or aromatic (e.g., heteroaryl) monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, acylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and loweralkyl above.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Arylalkenyl” as used herein alone or as part of another group, refers to an aryl group, as defined herein, appended to the parent molecular moiety through an alkenyl group, as defined herein.

“Arylalkynyl” as used herein alone or as part of another group, refers to an aryl group, as defined herein, appended to the parent molecular moiety through an alkynyl group, as defined herein.

“Heteroaryl” as used herein is as described in connection with heterocyclo and aryl above.

“Heteroalkyl” as used herein by itself or in combination with another term, means, unless otherwise stated, a stable straight or branched chain, or cyclic hydrocarbon radical (e.g., “heterocycloalkyl” or “heteroarylalkyl”), or combinations thereof, comprising an alkyl group, as defined herein, and at least one heteroatom selected from the group consisting of O, N, and S, and wherein the nitrogen, carbon and sulfur atoms may optionally be oxidized and the nitrogen heteroatom may optionally be quaternized. The heteroatom(s) O, N and S may be placed at any interior position of the alkyl group or at the position at which the alkyl group is attached to the remainder of the molecule. Examples include, but are not limited to, —CH₂—CH₂—O—CH₃, —CH₂—CH₂—O—CH₂—CH₃, —CH₂—O—CH₂—CH₂—O—CH₃, —O—CH₂—CH₂—O—CH₂—CH₃, —O—CH₂—O—CH₃, —O—CH₂—O—CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, and —CH₂—CH₂—S(O)₂—CH₃. Up to two heteroatoms may be consecutive, such as, for example, —CH₂—NH—OCH₃. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

“Heteroalkylene” as used herein by itself or in combination with another term means, unless otherwise stated, a stable straight or branched chain, or cyclic hydrocarbon radical (e.g., “heterocycloalkenyl” or “heteroarylalkenyl”), or combinations thereof, comprising an alkenyl group, as defined herein, and at least one heteroatom selected from the group consisting of O, N, and S, and wherein the nitrogen, carbon and sulfur atoms may optionally be oxidized and the nitrogen heteroatom may optionally be quaternized. The heteroatom(s) O, N and S may be placed at any interior position of the alkenyl group or at the position at which the alkenyl group is attached to the remainder of the molecule. For heteroalkylene groups, heteroatoms can also occupy either or both of the chain termini (e.g., alkyleneoxy, alkylenedioxy, alkyleneamino, alkylenediamino, and the like). Exemplary heteroalkylenes include, but not limited to, —CH═CH—O—CH₃, —CH₂—CH═N—OCH₃, and —CH═CH—N(CH₃)—CH₃. The terms “heteroalkyl” and “heteroalkylene” encompass poly(ethylene glycol) and its derivatives (see, for example, Shearwater Polymers Catalog, 2001). Still further, for alkylene and heteroalkylene linking groups, no orientation of the linking group is implied by the direction in which the formula of the linking group is written. For example, the formula —C(O)₂R′ represents both —C(O)₂R′ and —R′C(O)₂.

“Heteroalkynyl” as used herein by itself or in combination with another term means, unless otherwise stated, a stable straight or branched chain, or cyclic hydrocarbon radical (e.g., “heterocycloalkynyl” or “heteroarylalkynyl”), or combinations thereof, comprising an alkynyl group, as defined herein, and at least one heteroatom selected from the group consisting of O, N, and S, and wherein the nitrogen, carbon and sulfur atoms may optionally be oxidized and the nitrogen heteroatom may optionally be quaternized. The heteroatom(s) O, N and S may be placed at any interior position of the alkynyl group or at the position at which the alkenyl group is attached to the remainder of the molecule.

“Alkoxy” as used herein alone or as part of another group, refers to an alkyl or loweralkyl group, as defined herein (and thus including substituted versions such as polyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Aryloxy” as used herein alone or as part of another group refers to an aryl group, as defined herein (and thus including substituted versions), appended to the parent molecular moiety through an oxy group, —O—.

“Hydroxyalkyl” as used herein alone or as part of another group refers to a hydroxyl group, as defined herein, appended to the parent molecular moiety through an alkyl group as defined herein (and thus including substituted versions). Representative examples of hydroxyalkyl include, but are not limited to, hydroxymethyl, hydroxyethyl, hydroxypropyl and the like.

“Dihydroxyalkyl” as used herein refers to two hydroxyl groups, as defined herein, appended to the parent molecular moiety through an alkyl group, as defined herein (and thus including substituted versions). The hydroxyl groups may be attached to the same carbon atom or different carbon atoms of the alkyl group.

“Halo” as used herein refers to any suitable halogen, including F, Cl, Br and I.

“Mercapto” as used herein refers to an —SH group.

“Azido” as used herein refers to an —N₃ group.

“Cyano” as used herein refers to a —CN group.

“Formyl” as used herein refers to a —C(O)H group.

“Carboxylic acid” or “carboxy” as used herein alone or as part of another group, refers to a —C(O)OH group.

“Hydroxyl” as used herein alone or as part of another group, refers to an —OH group.

“Nitro” as used herein refers to an —NO₂ group.

“Oxo” as used herein, refers to a ═O moiety.

“Acyl” as used herein alone or as part of another group refers to a —C(O)R radical, where R is any suitable substituent such as aryl, alkyl, alkenyl, alkynyl, cycloalkyl or other suitable substituent as described herein.

“Carboxyacyl” as used herein refers to a carboxylic acid, as defined herein, appended to the parent molecular moiety through an acyl group, as defined herein. Representative examples of carboxyacyl include, but are not limited to, the following:

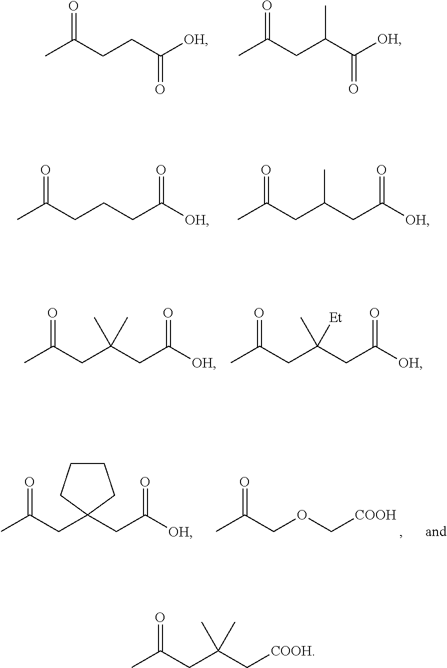

“Carboxyhydroxyalkyl” as used herein refers to a carboxy group, as defined herein, appended to the parent molecular moiety through an alkyl group, as defined herein, that is substituted with one or more hydroxy groups, as defined herein. The one or more hydroxy groups may be attached to the same carbon atom or different carbon atoms of the alkyl group and the alkyl group may be further substituted, as defined herein.

“Alkylthio” as used herein alone or as part of another group, refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Amino” as used herein means the radical —NH₂.

“Alkylamino” as used herein alone or as part of another group means the radical —NHR, where R is an alkyl group.

“Dialkylamino” as used herein refers to the radical —NRR′, where R and R′ are alkyl groups, as defined herein.

“Disubstituted amino” as used herein refers to the radical —NRR′, where R and R′ are substituents, as defined herein and may be the same or different.

“Acetyl” as used herein refers to the radical —C(═O)CH₃.

“Acetamidoalkyl” as used herein refers to a group of the structure

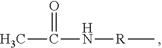

where R is appended to the parent molecular moiety and is an alkyl group, as defined herein.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and R_{b} is selected from the groups hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical —OR, where R is an acyl group as defined herein.

“Ester” as used herein alone or as part of another group refers to a —C(O)OR radical, where R is any suitable substituent such as alkyl, cycloalkyl, alkenyl, alkynyl or aryl.

“Amide” as used herein alone or as part of another group refers to a —C(O)NRₐR_{b} radical, where Rₐ and R_{b} are any suitable substituent such as alkyl, cycloalkyl, alkenyl, alkynyl or aryl.

“Sulfoxyl” as used herein refers to a compound of the formula —S(O)R, where R is any suitable substituent such as alkyl, cycloalkyl, alkenyl, alkynyl or aryl.

“Sulfonyl” as used herein refers to a compound of the formula —S(O)(O)R, where R is any suitable substituent such as alkyl, cycloalkyl, alkenyl, alkynyl or aryl.

“Sulfonate” as used herein refers to a compound of the formula —S(O)(O)OR, where R is any suitable substituent such as alkyl, cycloalkyl, alkenyl, alkynyl or aryl.

“Sulfonic acid” as used herein refers to a compound of the formula —S(O)(O)OH.

“Sulfonamide” as used herein alone or as part of another group refers to a —S(O)₂NRₐR_{b} radical, where Rₐ and R_{b} are any suitable substituent such as H, alkyl, cycloalkyl, alkenyl, alkynyl or aryl.

“Urea” as used herein alone or as part of another group refers to an —N(R_{c})C(O)NRₐR_{b} radical, where Rₐ, R_{b} and R_{c} are any suitable substituent such as H, alkyl, cycloalkyl, alkenyl, alkynyl or aryl.

“Alkoxyacylamino” as used herein alone or as part of another group refers to an —N(Rₐ)C(O)OR_{b} radical, where Rₐ, R_{b} are any suitable substituent such as H, alkyl, cycloalkyl, alkenyl, alkynyl or aryl.

“Aminoacyloxy” as used herein alone or as part of another group refers to an —OC(O)NRₐR_{b} radical, where Rₐ and R_{b} are any suitable substituent such as H, alkyl, cycloalkyl, alkenyl, alkynyl or aryl.

“Peptide” as used herein refers to a polymer of 2, 3 or 4 or more, up to 5 or 10, aminocarboxylic acid (or amino acid) monomers linked to one another by peptide bonds. When a substituent or radical, the polypeptide may be coupled to the parent molecule by its caroboxy terminus or its amino terminus. The individual amino acids may be natural or synthetic, standard or rare, and in the D or L configuration. Examples of individual amino acids include but are not limited to alanine, valine, leucine, isoleucine, glycine, serine, threonine, methionine, cysteine, phenylalanine, tyrosine, tryptophan, aspartic acid, glutamic acid, lysine, arginine, histidine and proline.

“Polyalkylene oxide” as used herein are known (see, e.g., U.S. Pat. No. 7,462,687) and include poly(ethylene glycol) or “PEG”. Additional examples may contain hetero atoms such as S or N, and are typically linear polyalkylene oxides such as: O—(CH₂CH₂O)ₓ—, —O—C(O)CH₂—O—(CH₂CH₂O)ₓ—CH₂C(O)—O—, —NRCH₂CH₂2-O—(CH₂CH₂O)ₓ—CH₂CH₂NR—, and —SHCH₂CH₂—O—(CH₂CH₂O)ₓ, —CH₂CH₂SH—, wherein R is H or loweralkyl (preferably methyl), and x is an integer of from about 1 to 6 or 10. Thus the polyalkylene oxide typically has a total number average molecular weight of from about 50 to 300 Daltons.

“Pharmaceutically acceptable” as used herein means that the compound or composition is suitable for administration to a subject to achieve the treatments described herein, without unduly deleterious side effects in light of the severity of the disease and necessity of the treatment.

A “prodrug” as used herein means a compound that is converted under physiological conditions or by solvolysis or metabolically to a specified compound that is pharmaceutically active.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

“Stereoisomer” as used herein refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

“Tautomer” as used herein refers to a proton shift from one atom of a molecule to another atom of the same molecule. As one skilled in the art would recognize tautomers often exist in equilibrium with each other and can interconvert under environmental and physiological conditions providing the same useful biological effects. Thus, the present invention includes mixtures of such tautomers. Additionally, a single compound may exhibit more than one type of isomerism. The present invention includes tautomers of any said active compounds of the present invention.

“Treat” as used herein refers to any type of treatment that imparts a benefit to a patient afflicted with a disease, including improvement in the condition of the patient (e.g., in one or more symptoms), delay in the progression of the disease, etc.

“Concurrently administer” as used herein means that the two compounds or agents are administered closely enough in time to produce a combined effect (that is, concurrently may be simultaneously, or it may be two or more events occurring within a short time period before or after each other, e.g., sequentially). Simultaneous administration may be carried out by mixing the compounds prior to administration, or by administering the compounds at the same point in time but at different anatomic sites and/or by using different routes of administration.

All publications, U.S. patent applications, U.S. patents and other references cited herein are incorporated by reference in their entireties.

1. Active Compounds.

The methods of the present invention include the administration of active compounds as described herein (e.g., compounds of Formula (I)), while pharmaceutical compositions of the present invention comprise active compounds in a pharmaceutically acceptable carrier or diluent.

Active compounds of Formula (I) are:

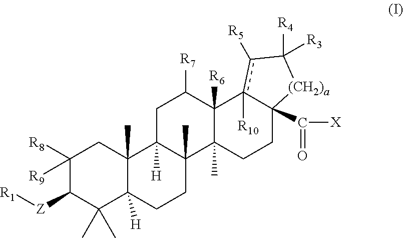

wherein:

a is 1 or 2;

Z is O, S, NH, or N-alkyl;

R₁ is a hydrogen, acyl carboxylic acid, C₂ to C₂₀ substituted or unsubstituted carboxyacyl, or a substituent of the formula:

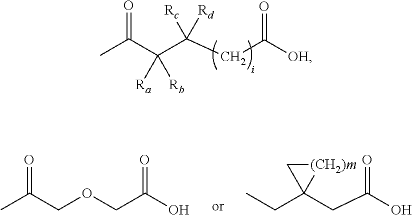

wherein Rₐ, R_{b}, R_{c} and R_{d} are the same or different and are each independently selected from the group consisting of hydrogen and lower alkyl, i is an integer from 0 to 3, and m is an integer from 1 to 4;

X is polyalkylene oxide, heteroalkylene, or —NR₂ₐR_{2b}, wherein R₂ₐ is H, loweralkyl, heteroalkylene, or polyalkylene oxide and R_{2b} is H, heteroalkylene, polyalkylene oxide, or a substituent of the formula:

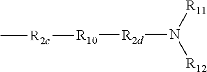

where R_{2c} is C2 to C10 saturated or unsaturated alkylene, R_{2d} is present or absent and when present is C1 to C5 saturated or unsaturated alkylene, R₁₀ is CONH, NHCO, NH, SH, or O, and R₁₁ and R₁₂ are each H, loweralkyl, heteroalkyl, carboxy, amino acid, or a peptide, or R₁₁ and R₁₂ together form with the N to which they are joined cycloalkyl or heterocycloalkyl;

or R₂ₐ and R_{2b} together are C3 to C5 alkylene, which alkylene is substituted or unsubstituted;

R₃ and R₄ are either H or lower alkyl (e.g., methyl);

R₅ is H, lower alkyl, or —CRᵢRᵢᵢRᵢᵢᵢ, where:
Rᵢ is a methyl radical or forms with Rᵢᵢ a methylene radical or an oxo radical;
Rᵢᵢ is a hydrogen atom or forms with Rᵢ or Rᵢᵢᵢ a methylene radical or an oxo radical; and
Rᵢᵢᵢ is a hydroxyl, methyl, hydroxymethyl, —CH₂OR′ᵢᵢᵢ, —CH₂SR′ᵢᵢᵢ, or —CH₂NHR′ᵢᵢᵢ, which R′ᵢᵢᵢ is alkyl, hydroxyalkyl, dihydroxyalkyl, acetamidoalkyl, acetyl, heteroalkylene, or polyalkylene oxide; or Rᵢᵢᵢ is an amino radical substituted with hydroxyalkyl, carboxyhydroxyalkyl, or dialkylamino, the alkyl parts of which can form, with the nitrogen atom to which they are joined, a 5- or 6-membered heterocycle optionally containing 1 or 2 additional hetero atoms selected from the group consisting of O, S, NH, and N-alkyl;
   or R₅ is a bond to an immediately adjacent carbon atom (thus forming a double bond in the ring between immediately adjacent carbon atoms);

R₆ and R₇ are either H or form a bond with one another (thus forming a double bond between their immediately adjacent carbon atoms);

R₈ and R₉ are either hydrogen or together form an oxo radical;

R₁₀ is either H or a bond with an immediately adjacent carbon atom (thus forming a double bond in the ring between immediately adjacent carbon atoms); and

the dashed line in Formula (I) is an optional double bond;

or a stereoisomer, enantiomer, tautomer thereof or mixtures thereof;
or a pharmaceutically acceptable salt or prodrug thereof.

In some embodiments of Formula (I), X is —NR₂ₐR_{2b} and R_{2b} is a substituent of the formula:

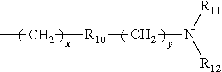

where x is an integer from 2 to 10, y is an integer from 0 to 5.

In some embodiments of Formula (I), X is —NR₂ₐR_{2b} and R₂ₐ and R_{2b} together form a substituent of the formula:

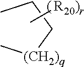

wherein:

q is 1, 2, or 3;

r is 1, 2 or 3; and

each R₂₀ is independently selected from the group consisting of alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, aryloxy, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxyacylamino, and aminoacyloxy.

In some embodiments of the foregoing, R₂₀ is a substituent of the formula:

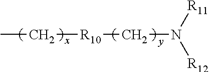

where x is an integer from 1, 2 or 3 to 8 or 10, y is an integer from 0 or 1 to 5, R₁₀ is CONH, NHCO, NH, SH, or O, and R₁₁ and R₁₂ are each H, loweralkyl, heteroalkyl, carboxy, amino acid, or a peptide, or R₁₁ and R₁₂ together form with the N to which they are joined cycloalkyl or heterocycloalkyl.

In some embodiments of Formula (I), X is heteroalkylene or polyalkylene oxide.

In some embodiments of Formula (I), R₁ is C₂ to C₂₀ substituted or unsubstituted carboxyacyl.

In some embodiments of Formula (I), R₁ contains at least one asymmetric center with a (S) configuration.

In some embodiments of Formula (I), X is heteroalkylene, Z is O, and R1 is C₂ to C₂₀ substituted or unsubstituted carboxyacyl.

In some embodiments of Formula (I) the active compound has the structure:

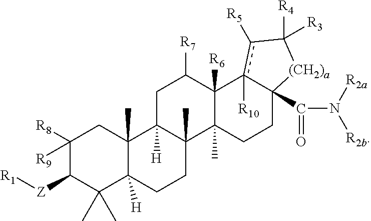

In some embodiments of Formula (I), R₁ is a hydrogen, or a substituent of the formula:

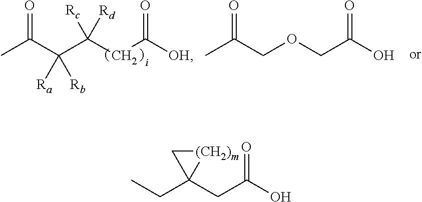

wherein Rₐ, R_{b}, R_{c} and R_{d} are the same or different and are each independently selected from the group consisting of hydrogen or lower alkyl, i is an integer from 0 to 3, and m is an integer from 1 to 4.

In some embodiments of Formula (I), R₆ and R₁₀ are each H.

In some embodiments of Formula (I), R₁ is

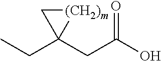

and m is an integer from 1 to 4.

In some embodiments of Formula (I), R₅ is —CRᵢRᵢᵢRᵢᵢᵢ.

In some embodiments of Formula (I), R₈ and R₉ are each H.

In some embodiments of Formula (I), Rᵢ and Rᵢᵢᵢ together form a methylene radical.

In some embodiments of Formula (I), Rᵢᵢ is methyl.

In some embodiments of Formula (I), R₃ and R₄ are each H.

In some embodiments of Formula (I), R₆ and R₇ are each H.

Non-limiting examples of compounds of Formula (I) where X is —NR₂ₐR_{2b} which incorporate an unsaturated chain at the R₂, or R_{2d} position are:

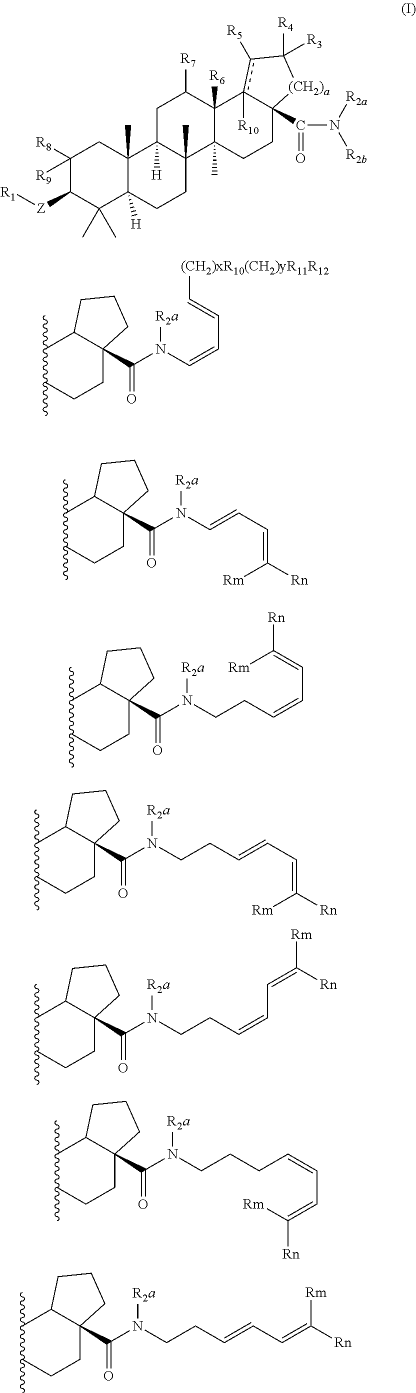

where Rm and Rn could be H or (CH₂)xR₁₀(CH₂)_{y}R₁₁R₁₂

The active compounds disclosed herein or described above can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Active compounds may be provided as pharmaceutically acceptable prodrugs, which are those prodrugs of the active compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

2. Pharmaceutical Formulations

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter glia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a compound of Formula (I), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 1 or 10 mg to about 100 milligrams, 1 gram or 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

In addition to compounds of Formula (I) or their salts, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

3. Methods of Treatment

The present invention is primarily concerned with the treatment of human subjects, but the invention may also be carried out on animal subjects, particularly mammalian subjects such as mice, rats, dogs, cats, non-human primates, livestock and horses for veterinary purposes, and for drug screening and drug development purposes.

Examples of retroviral infections that may be treated by the methods of the present invention include but are not limited to feline leukemia virus (FeLV), human immunodeficiency virus (HIV; including both HIV-1 and HIV-2) simian immunodeficiency virus (SIV) and other lentiviral infections such as equine infectious anemia virus (EAIV) and feline immunodeficiency virus (FIV). A particularly preferred embodiment is use of the methods, compounds and compositions of the present invention for the treatment of HIV-1 infection in human subjects.

4. Dosage and Routes of Administration

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any one active agent, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art.

Typical dosages comprise at about 0.1 to about 100 mg/kg body weight. One preferred dosages comprise about 1 to about 100 mg/kg body weight of the active ingredient. One still more preferred dosages comprise about 10 to about 100 mg/kg body weight.

5. Combination Methods and Compositions

Methods of treatment as described herein can include concurrently administering one or more additional antiviral agent (including HIV entry inhibitors as discussed below), and compositions as described herein can optionally include one or more such additional antiviral agents. Examples of such additional antiviral agents include, but are not limited to, AZT (Glaxo Wellcome), 3TC (Glaxo Wellcome), ddI (Bristol-Myers Squibb), ddC (Hoffmann-La Roche), D4T (Bristol-Myers Squibb), abacavir (Glaxo Wellcome), nevirapine (Boehringher Ingelheim), delavirdine (Pharmacia and Upjohn), efavirenz (DuPont Pharmaceuticals), saquinavir (Hoffmann-La Roche), ritonavir (Abbott Laboratories), indinavir (Merck and Company), nelfinavir (Agouron Pharmaceuticals), amprenavir (Glaxo Wellcome), adefovir (Gilead Sciences), hydroxyurea (Bristol-Meyers Squibb), AL-721 (lipid mixture) manufactured by Ethigen Corporation and Matrix Research Laboratories; Amphotericin B methyl ester; Ampligen (mismatched RNA) developed by DuPont/HEM Research; anti-AIDS antibody (Nisshon Food); 1 AS-101 (heavy metal based immunostimulant); Betaseron (.beta.-interferon) manufactured by Triton Biosciences (Shell Oil); butylated hydroxytoluene; Carrosyn (polymannoacetate); Castanospermine; Contracan (stearic acid derivative); Creme Pharmatex (containing benzalkonium chloride) manufactured by Pharmalec; CS-87 (5-unsubstituted derivative of Zidovudine), Cytovene (ganciclovir) manufactured by Syntex Corporation; dextran sulfate; D-penicillamine (3-mercapto-D-valine) manufactured by Carter-Wallace and Degussa Pharmaceutical; Foscarnet (trisodium phosphonoformate) manufactured by Astra AB; fusidic acid manufactured by Leo Lovens; glycyrrhizin (a constituent of licorice root); HPA-23 (ammonium-21-tungsto-9-antimonate) manufactured by Rhone-Poulenc Sante; human immune virus antiviral developed by Porton Products International; Ornidyl (eflornithine) manufactured by Merrell-Dow; nonoxinol; pentamidine isethionate (PENTAM-300) manufactured by Lypho Med; Peptide T (octapeptide sequence) manufactured by Peninsula Laboratories; Phenyloin (Warner-Lambert); Ribavirin; Rifabutin (ansamycin) manufactured by Adria Laboratories; CD4-IgG2 (Progenies Pharmaceuticals) or other CD4-containing or CD4-based molecules; T-20 (Trimeris); Trimetrexate manufactured by Warner-Lambert Company; SK-818 (germanium-derived antiviral) manufactured by Sanwa Kagaku; suramin and analogues thereof manufactured by Miles Pharmaceuticals; UA001 manufactured by Ueno Fine Chemicals Industry; and alpha-interferon, manufactured by Glaxo Wellcome.

HIV entry inhibitors are a class of anti HIV drugs that work by preventing HIV from entering susceptible cells in the body. In generally, it is preferred that the HIV entry inhibitor (1) block virus entry into susceptible cells by preventing HIV-1 binding to the cellular receptor CD4, the coreceptors CXCR4/CCR5 and to receptors on dendritic/migratory cells (capturing and transmitting virus to cells which are directly involved in virus replication), respectively. (See The entry of entry inhibitors: a fusion of science and medicine, Moore, J. P, etc, Proc. Natl. Acad. Sci., USA, 100, 10598-10602, (2003); HIV-1 entry inhibitors: new targets, novel therapies, Pierson, T. C., etc., Immunol. Lett., 85, 113-118, (2003); HIV Transmission: Closing all the Doors, Davis, C. W., etc, J. Exp. Med., 199, 1037-1040, (2004); Blockade of attachment and fusion receptors inhibits HIV-1 infection of human cervical tissue, Hu, Q., Frank, etc, J. Exp. Med., 199, 1065-1075, (2004)), and/or (2) are virucidal.)

Examples of HIV inhibitors include but not limited to: CCR5 inhibitors TAK-779, Fusion inhibitors T20, CXCR4 inhibitor AMD 3100, and other inhibitors BMS 378806, etc. The structures of representative HIV entry inhibitors are illustrated below.

TAK-779 is also known as N,N-dimethyl-N-(4[[[2-(4-methylphenyl)-6,7-dihydro-5H-benzocyclohepten-8-yl]carbon-yl]benzyl]-tetrahydro-2H-pyran (See Structure Modeling of the Chemkine Receptor CCR5: Implications for Ligand Binding and Selectivity, M. Germana Paterlini, Biophysical Journal, 83, 3012-3031 (2002).)

T20 is also known as enfuviritide and is commercially available as FUZEON™. It is a linear 36-amino acid synthetic peptide with an acetylated N-terminus and a carboxamide C-terminus. It is composed of naturally occurring L-amino acid residues. The empirical formula of enfuvirtide is C₂₀₄H₃₀₁N₅₁O₆₄. It has the following primary amino acid sequence: CH₃CO-Tyr-Thr-Ser-Leu-Ile-His-Ser-Leu-Ile-Glu-Glu-Ser-Gln-Asn-Gln-Gln-Glu-Lys-Asn-Glu-Gln-Glu-Leu-Leu-Glu-Leu-Asp-Lys-Trp-Ala-Ser-Leu-Trp-Asn-Trp-Phe-NH2.

BMS-378806 is also known as (2R)-4-benzoyl-1-[2-(4-methoxy-1H-pyrrolo[2,3-b]pyridin-3-yl)-1,2-dioxoethyl]-2-methyl-piperazine (See A small molecule HIV-1 inhibitor that targets the HIV-1 envelope and inhibits CD4 receptor binding, Pin-Fang Lin, et al., PNAS, 100, 19, 1103-11018 (2003, September)).

AMD 3100, is also known as 1,1′-[1,4-phenylenebis(methylene)]-bis-1,4,8,11-tetraazacyclotetradecane is shown below. (See AMD 3100, a Potent and Specific Antagonist of the Stromal Cell-Derived Factor-1 Chemokine Receptor CXCR4, Inhibits Autoimmune Joint Inflammation in IFN-γ Receptor-Deficient Mice, Patrick Matthys, etc, The Journal of Immunology, 167, 4686-4692. (2001).)

The compounds of the present invention may be concurrently administered in combination with one or more HIV entry inhibitors for the treatment of a mammal, such as a human, that is suffering from an infection with the HIV virus, AIDS, AIDS-related complex, or any disease or condition which is related to infection with the HIV virus.

Pharmaceutical compositions of the present invention can also further comprise immunomodulators, and methods of treatment of the present invention can include the co-administration of an immunomodulator. Suitable immunomodulators for optional use with the active compounds of the present invention in accordance with the present invention can include, but are not limited to: ABPP (Bropririmine); Ampligen (mismatched RNA) DuPont/HEM Research; anti-human interferon-.alpha.-antibody (Advance Biotherapy and Concepts); anti-AIDS antibody (Nisshon Food); AS-101 (heavy metal based immunostimulant; ascorbic acid and derivatives thereof; interferon-.beta.; Carrosyn (polymannoacetate); Ciamexon (Boehringer-Mannheim); cyclosporin; cimetidine; CL-246,738 (American Cyanamid); colony stimulating factors, including GM-CSF (Sandoz, Genetics Institute); dinitrochlorobenzene; HE2000 (Hollis-Eden Pharmaceuticals); interferon-.alpha.; interferon-gamma; glucan; hyperimmune gamma-globulin (Bayer); IMREG-1 (leukocyte dialyzate) and IMREG-2 (IMREG Corp.); immuthiol (sodium diethylthiocarbamate) (Institut Merieux); interleukin-1 (Cetus Corporation; Hoffmann-LaRoche; Immunex); interleukin-2 (IL-2) (Chiron Corporation); isoprinosine (inosine pranobex); Krestin (Sankyo); LC-9018 (Yakult); lentinan (Ajinomoto/Yamanouchi); LF-1695 (Fournier); methionine-enkephalin (TNI Pharmaceuticals; Sigma Chemicals); Minophagen C; muramyl tripeptide, MTP-PE (Ciba-Geigy); naltrexone (“Trexan” DuPont); Neutropin, RNA immunomodulator (Nippon Shingaku); Remune (Immune Response Corporation); Reticulose (Advanced Viral Research Corporation); shosaikoto and ginseng; thymic humoral factor; TP-05 (Thymopentin, Ortho Pharmaceuticals); Thymosin factor 5 and Thymosin 1; Thymostimulin; TNF (Tumor necrosis factor) manufactured by Genentech; and vitamin B preparations.

Further embodiments of the present invention will now be described with reference to the following examples. It should be appreciated that these examples are for the purposes of illustrating embodiments of the present invention, and do not limit the scope of the invention.

### EXAMPLES

### Example 1

The high potency and novel mechanism of 3a suggest that further modification of this compound class as HIV entry inhibitors is warranted. Therefore, in the current study, we mainly focused on the modification of BA to maintain the anti-HIV activity while improving pharmacokinetic properties.

Design. Structurally, the triterpenoid skeleton of BA contains three functional groups: C-3 hydroxyl group, C-28 carboxylic acid group and C-19 isopropenyl moiety. Because the C-19 isopropenyl group has been less investigated, modification was first carried out on this moiety. Previous research suggested that saturation of the 20(29) double bond did not influence the antiviral activity of the BA derivatives significantly.¹⁴ Therefore, we focused on the modification at the C-30 alkyl position in order to better explore the SAR. Some early data suggested that thioether-linked substitution on the C-30 position retained the anti-HIV-1 potency slightly in the resulting BA analogs.²⁶ In the current study, the bioisosteric oxygen ether linker was chosen to replace the thioether group. Leucine and 8-aminooctanoic acid were proved to improve entry inhibition in our previous study, thus they were incorporated into the C-28 side chain of the diverse C-30 modified analogs to yield compounds 10-30.

The fact that 2 has suitably pharmocokinetic properties for development, but 3a does not, led us to speculate that the C-28 side chain, which is critical for anti-HIV entry activity, may be responsible for the poor performance of 3a in pre-clinical testing. Indeed, in our study, we observed that C-28 modified BA derivatives, including A43-D (4) which is the prior best anti-entry hit, are less water-soluble. In the meanwhile, although 2 has been demonstrated to be metabolized primarily by UGT glucuronidation,²⁷ there is no report regarding to the metabolism of C-28 modified BA analogs. Therefore, in the present study, in vitro metabolic stability assessment was first carried out in pooled human liver microsomes (BD Biosciences), which contain enzymes including cytochrome P450 (CYPs), UGTs, FMO and OR, etc. The results revealed that C-28 modified BA derivatives like A43-D (4) are fast metabolized in liver microsomes. Therefore, novel C-28 side chains were designed and synthesized. A cyclic secondary amine (from 4-piperidine butyric acid), rather than a primary amine, was used to form the critical amide bond with the C-28 carboxylic acid group, which yielded 38.

Moreover, although BA derivatives with both C-3 ester and C-28 amide side chains exhibit both anti-entry and maturation activity,²⁸ they also display some metabolic problems, likely due to the C-28 side chain. Thus, modifications were also carried out on C-28 and C-3 side chains to enhance the stability as well as increase the anti-HIV activity in the 3,28-disubstituted BA analogs, which yielded compounds 36, 39-40 and 45-48. This article reports the design, syntheses, SAR and metabolic stability assessment of these novel BA derivatives as potent HIV-1 inhibitors.

### Chemistry

Scheme 1 depicts the synthetic pathway of 28,30-disubstituted BA derivatives 10-30. The C-3β-hydroxyl group of BA was protected as the acetate ester of 3-O—Ac—BA (5). Compound 5 was reacted with oxalyl chloride in dichloromethane to yield an intermediate acid chloride, which was then treated with leucine methyl ester or 8-aminooctanoic acid methyl ester to furnish 6 and 7. Allylic bromination of 6 and 7 was carried out using N-bromosuccinimide (NBS) in dilute acetonitrile at room temperature to provide 30-bromo BA derivatives 8 and 9. The bromide group of 8 and 9 was then substituted by a diverse set of nucleophilic compounds to yield 10-20. In this step, the desired nucleophilic compound was first treated with 10 equivalents of NaH in THF for 30 min. The 30-bromo 8 or 9 was then reacted with the resulting suspension using a microwave apparatus at 120° C. After cooling down, 1 mL MeOH—H₂O was added to convert the intermediate esters to carboxylic acids by saponification, which furnished the target compounds 10-20 in 59-77% yields. Reaction of 20 with methylamine in the presence of HOBt/EDCI in dichloromethane led to 21 in a 69% yield. Saponification of the 30-bromo 8 and 9 with 2 N sodium hydroxide in MeOH/THF yielded the corresponding carboxylic acids 24-25. The previous reported 22-23 were also prepared by saponification of the ester intermediates 6 and 7. Reaction of silver acetate with 8 and 9, in the presence of a catalytic amount of the phase transfer catalyst tetrabutylammonium bromide (Bu₄NBr) in acetonitrile, gave diacetoxy esters 26 and 27, which were then converted to 30-hydroxyl BA derivatives 28 and 29. The 3,28,30-trisubstituted analog 30 was acquired by reaction of 24 with 2,2-dimethylsuccinic anhydride in the presence of DMAP in pyridine.

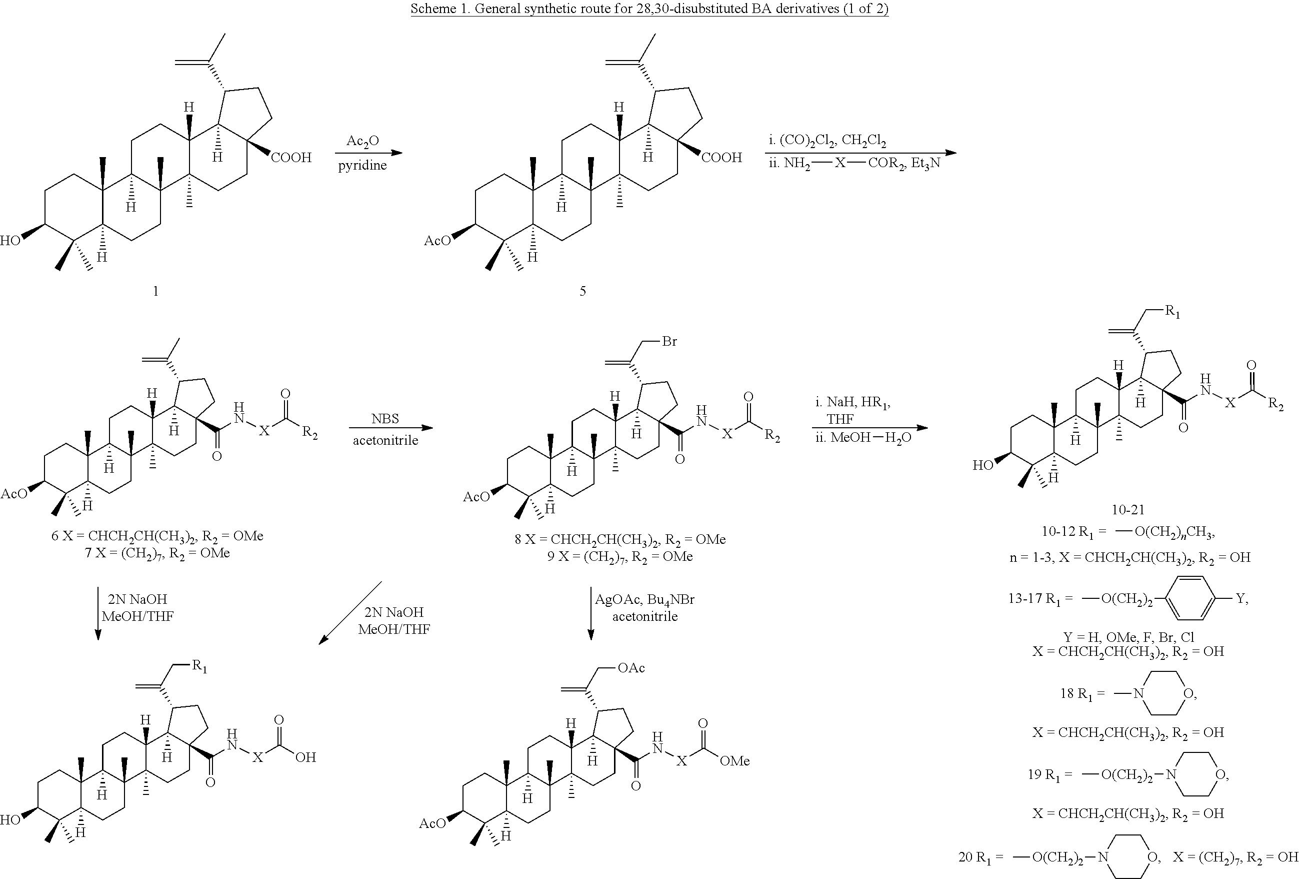

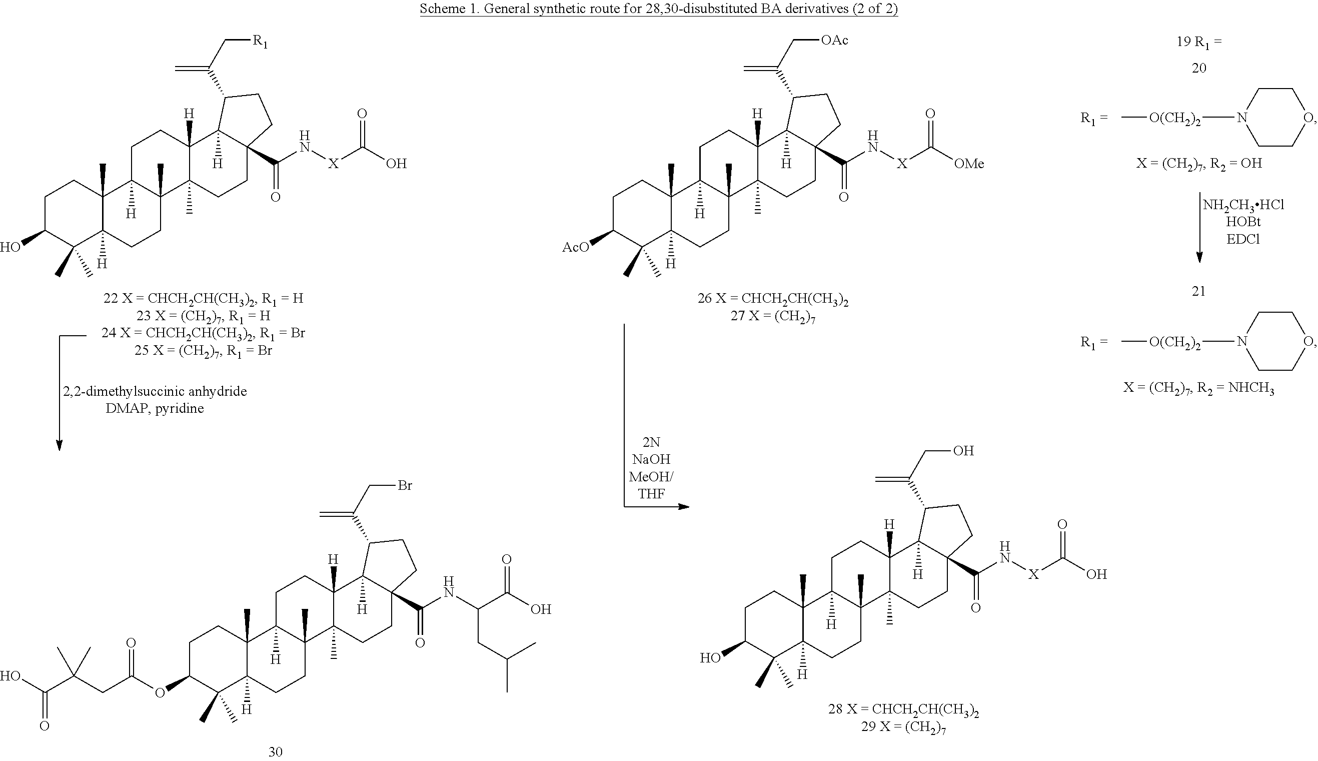

The 3,28-disubstituted 3β-amino analog 36 was successfully prepared as described in Scheme 2. Oxidation of 1 with 2 equivalents of PDC produced the 3-keto-BA 31 (87% yield). L-Leucine methyl ester was reacted with the C-28 carboxylic acid of 31 in the presence of DMAP and EDCI to furnish 32. The keto moiety of 32 was then converted to an oxime by treatment with hydroxylamine hydrochloride (NH₂OH.HCl) in pyridine, which yielded 33 (90% yield).²⁹ The 3β-amine 34 was readily prepared in 82% yield from oxime 33 by enantioselective reduction of the Schiff base with TiCl₃ and NaCNBH₃, as reported by Leeds and Kirst.³⁰ Treatment of 34 with 2,2-dimethylsuccinic anhydride under DMAP in pyridine yielded 35. Finally, hydrolysis of 35 with 2 N sodium hydroxide furnished the desired 3,28-disubstituted 3β-amino BA analog 36.

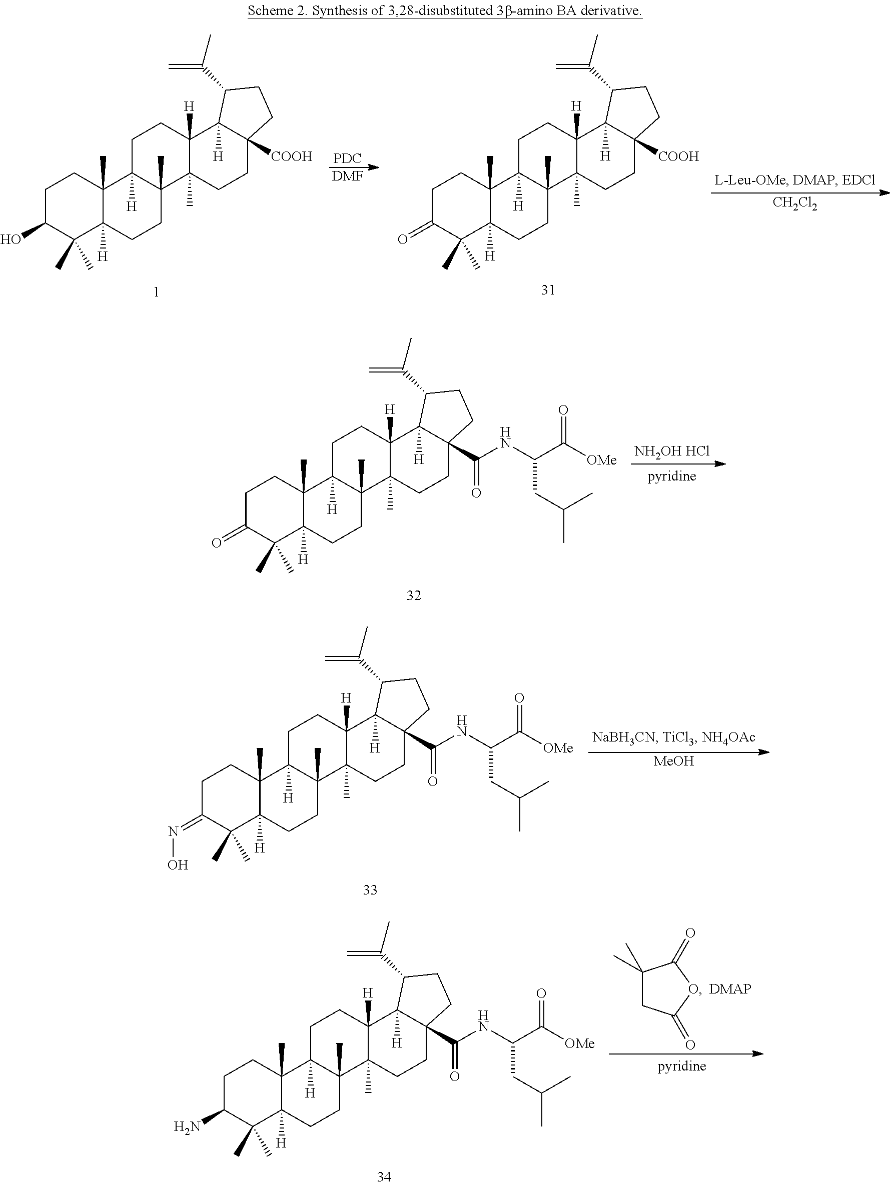

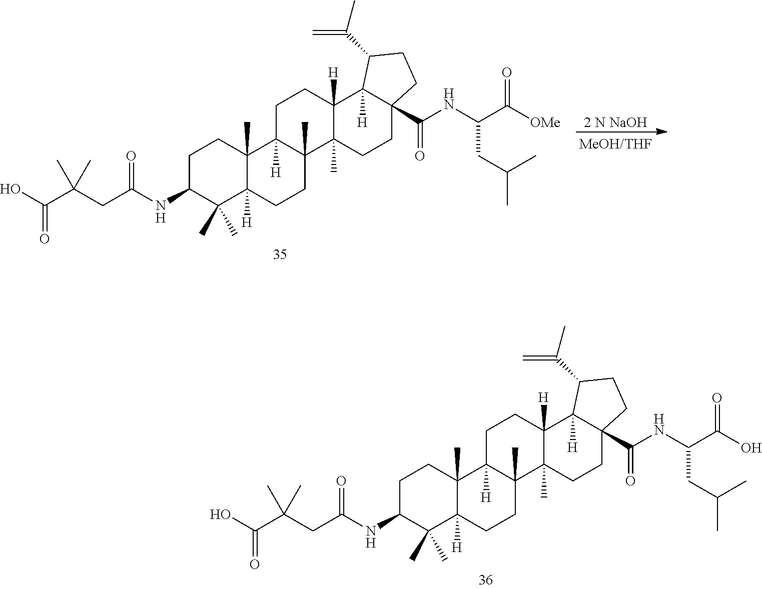

The syntheses of 3,28-disubstituted 28-piperidine analogs 38-48 were carried out according to Scheme 3. The 3-O—Ac—BA (5) was treated with oxalyl chloride in dichloromethane, followed by reaction with the readily prepared 4-piperidine butyric acid methyl ester to provide 37 in a 94% yield. Saponification of 37 yielded the desired lead compound 38 quantitatively. Esterification of 38 with 2,2-dimethylsuccinic anhydride and 2,2-dimethylglutaric anhydride under DMAP in pyridine led to 39 and 40 in yields of 55% and 36%, respectively. The syntheses of 41-44 were carried out by reacting 38 with different amines in the presence of HOBt and EDCI. Reaction of the 3β-hydroxyl group of 41-44 with 2,2-dimethylsuccinic anhydride provided the 3,28-disubstituted target compounds 45-48 in yields of 58-81%.0

### Results and Discussion

The anti-HIV-1 replication activities of the newly synthesized BA derivatives 10-30, 36, 38-40 and 45-48 were assessed in infected MT-2 lymphocytes in parallel with AZT and bevirimat (DSB, 2). Compounds 21 and 45-48 were further evaluated against HIV-1_{NL4-3} in MT-4 cell lines and compared with IC9564 (3b) and A43-D (4). Because these two antiviral screening systems used slightly different protocols, the results may vary for the same compounds. The bioassay data are summarized in Table 1 and 2, respectively.

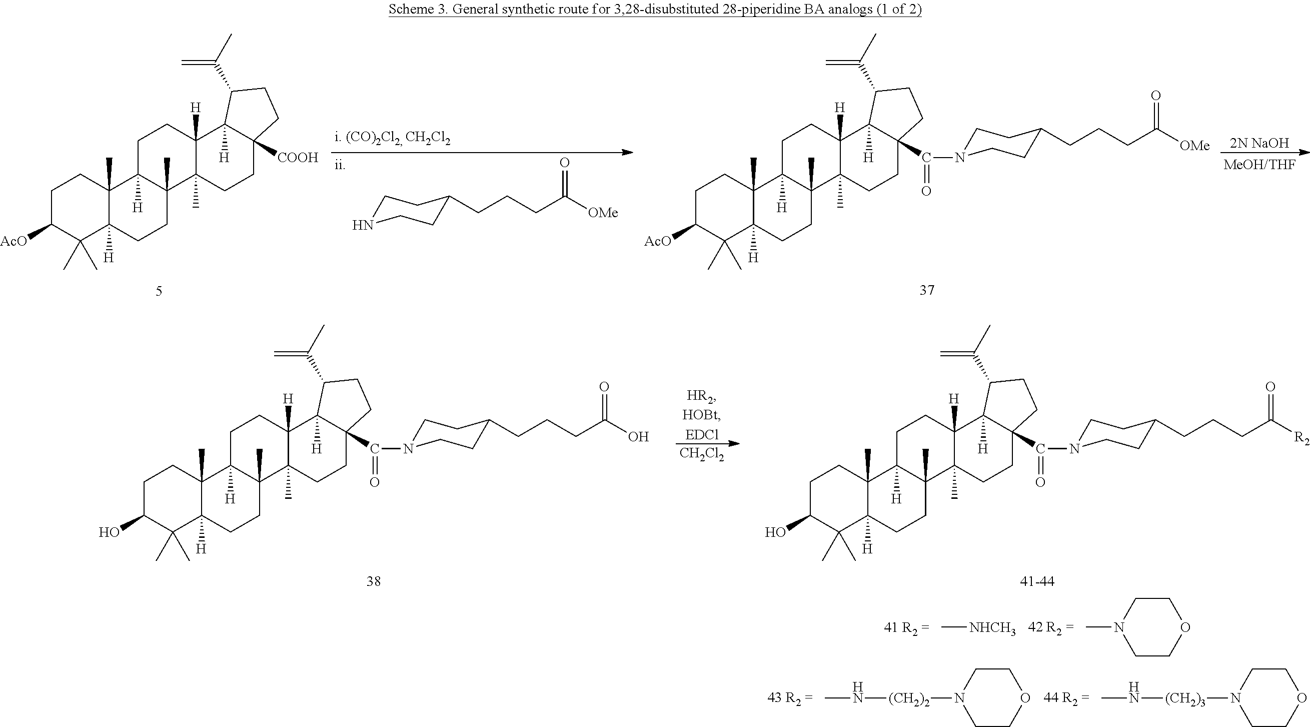

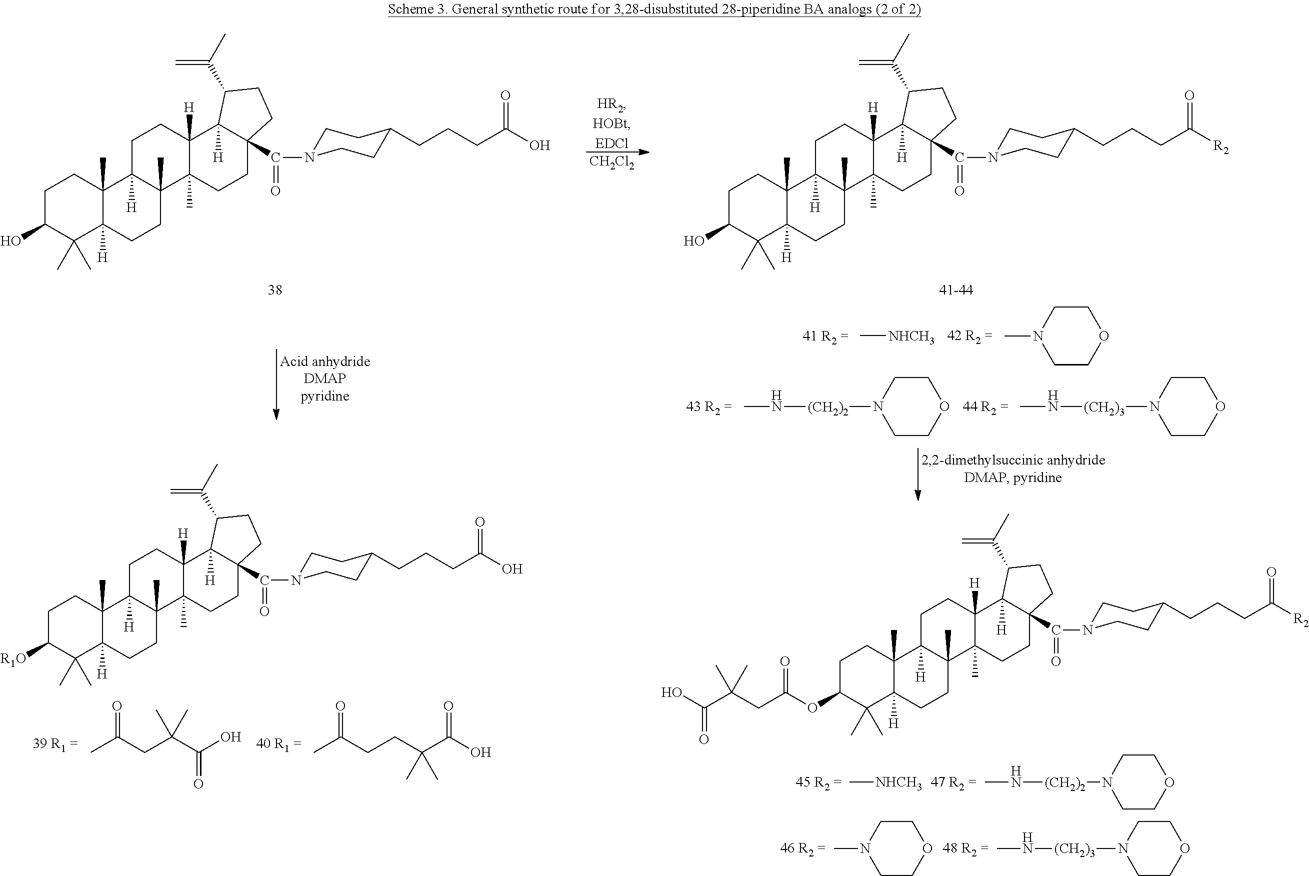

TABLE 1
Anti-HIV-1 replication activities in
HIV-1_{IIIB} infected MT-2 cell lines. ^{a}

| Compd. | EC₅₀ (μM) | CC₅₀ (μM) | TI |
|---|---|---|---|
| AZT | 0.056 | 1,870 | 33,392 |
| 2 | 0.011 | 40 | 3,636 |
| 10 | NS | 40.7 | — |
| 11 | NS | 25.3 | — |
| 12 | NS | 24.9 | — |
| 13 | NS | 21.7 | — |
| 14 | NS | 16.7 | — |
| 15 | NS | 24.4 | — |
| 16 | NS | 20.8 | — |
| 17 | 27.8 | 34.6 | 1.2 |
| 18 | NS | 27.5 | — |
| 19 | 26.6 | 35.8 | 1.4 |
| 20 | 1.8 | 24.8 | 13.8 |
| 21 | 0.09 | 22.3 | 250 |
| 22 | NS | 29.8 | — |
| 23 | 3.3 | 33.4 | 10.1 |
| 24 | NS | 26.8 | — |
| 25 | 2.3 | 36.9 | 16.1 |
| 28 | NS | 30.7 | — |
| 29 | 14.8 | 40.7 | 2.8 |
| 30 | 0.011 | 33.2 | 3,022 |
| 36 | 13.2 | 35.9 | 2.7 |
| 38 | 21.72 | 41.0 | 1.9 |
| 39 | 0.015 | 20.8 | 1,389 |
| 40 | 0.23 | 33.2 | 145 |
| 45 | 0.067 | 33.3 | 497 |
| 46 | 0.011 | 19.2 | 1,748 |
| 47 | 0.007 | 17.3 | 2,473 |
| 48 | 0.006 | 18.5 | 3,087 |

^{a} All data presented are averages of at least three separate experiments performed by Panacos Pharmaceuticals Inc., Gaithersburg, MD. EC₅₀: concentration that inhibits HIV-1_{IIIB} replication by 50%. CC₅₀: concentration that inhibits mock-infected MT-2 cell growth by 50%. TI = CC₅₀/EC₅₀. NS: no suppression at concentrations below the CC₅₀.

TABLE 2
Anti-HIV-1 replication activities in HIV-
1_{NL4-3} infected MT-4 cell lines. ^{b}

| Compd. | EC₅₀ (μM) | CC₅₀ (μM) | TI |
|---|---|---|---|
| 3b | 0.089 | >10 | >112.4 |
| 4 | 0.092 | >10 | >150 |
| 21 | 0.09 | >10 | >138 |
| 45 | 0.24 | >10 | >41.7 |
| 46 | 0.07 | 8.8 | 121.4 |
| 47 | 0.035 | 8.5 | 283.9 |
| 48 | 0.031 | 8.6 | 245.7 |

^{b} All data presented are averages of at least two separate experiments performed by Dr. Chin-Ho Chen, Duke University, NC. EC₅₀: concentration that inhibits HIV-1_{NL4-3} replication by 50%. CC₅₀: concentration that inhibits mock-infected MT-4 cell growth by 50%. TI = CC₅₀/EC₅₀. NS: no suppression at the testing concentration (10 μM).

Within the 28,30-disubstituted BA derivatives, the C-28 leucine modified 28,30-analogs 10-19, 24 and 28 did not inhibit viral replication. Since C-28 leucine substituted BA derivative 22 did not exhibit antiviral replication activity either, we postulate that it is not because of C-30 modifications that the resulting derivatives lose antiviral potency. However, the presence of different C-30 substitutions did not increase the anti-HIV-1 activity, suggesting that the C-19 isopropenyl moiety is unlikely to be an activity pharmacophore. Nevertheless, considering that BA derivatives with the anti-entry necessary C-28 lipophilic side chains are less water-soluble, C-30 allylic modification may still be useful to influence pharmacokinetic properties, such as hydrophilicity and solubility.

The introduction of a free hydroxyl group at C-30 reduced the antiviral activity of 29 by several fold to an EC₅₀ value of 14.8 μM. It suggested that a hydrogen bond donor is not tolerated near the C-19 isopropenyl group, which is also supported by previous data that a primary amine substituent at C-30 is unfavorable.²⁶ Compared to this, introduction of 2-morpholinoethoxy (20) and bromide moiety (25) keep the antiviral activity of 23. Specifically, the 28-aminooctanoic acid derivatives 23, 20 and 25 showed antiviral EC₅₀ values of 3.3 μM, 1.8 μM and 2.3 μM against HIV-1_{IIIB}, respectively. These results demonstrated that the C-30 position of BA can accommodate some diverse ethers without decreasing the anti-HIV potency. Moreover, the introduction of the morpholinoethoxy moiety in 20, which reduced the Log P value of 23 from 9.79 to 8.26 (calculated by ACD/LogP DB software), resulted in an increase in the derivative's solubility, confirming that the C-30 position may serve as a good place to incorporate water-solubilizing moieties.

Analog 21 with methylamine linked to the terminal carboxylic acid of 20 exhibited potent anti-HIV-1 activity with an EC₅₀ value of 0.09 μM and TI of 250 against both HIV-1_{IIIB} and HIV-1_{NL4-3} variants, which are similar to those of the prior best entry inhibitor 4 (EC₅₀: 0.10 TI>100) and comparable to those of AZT (EC₅₀: 0.056 μM, TI>3.3×10⁴). This result indicates that the amide moiety near the end of the C-28 side chain is necessary for enhanced antiviral potency. Interestingly, 21 differs from 4 in the direction of the terminal amide linkage (—CONHCH₃ in 21 and —NHCOCH₃ in 4). Compound 21 showed a significantly reduced Log P value of 7.5 compared with the lead compound 23 and prior best hit 4.

Analog 30 with 3′,3′-dimethylsuccinyl side chain linked to the C-3β-hydroxyl group of 24 showed extremely potent antiviral activity with an EC₅₀ value of 0.011 μM and TI of 3.0×10³, which are similar to those of 2 (EC₅₀: 0.011 μM, TI>3.6×10³) and slightly better than those of AZT. This result suggests that the presence of small substitutions on C-30 of BA does not harm the high anti-HIV-1 potency of 2. Thus, incorporation of polar groups into the C-30 position may also help to improve the hydrophilicity of 3,28-disubstituted BA derivatives.

Because an amide is generally more stable in vivo than an ester moiety, we also synthesized 3,28-disubstituted 3β-amino BA derived analog 36. Its C-3 side chain is similar to that of 2, except for a C-3 amide rather than ester bond. However, the antiviral activity of 36 against HIV-1_{IIIB} decreased significantly to an EC₅₀ value of 13.2 μM, suggesting that bioisosteric replacement of the C-3 ester bond with an amide moiety is not tolerated.

Results from the metabolism study revealed that changing the C-28 side chain from 8-aminooctanoic acid (23) to 4-piperidine butyric acid (38) could significantly increase the in vitro metabolic stability. Specifically, approximately 50% of 23 disappeared after around 35 minutes of incubation with pooled human liver microsomes, which is similar with buspirone (t_{1/2}=31 min), an established fast-metabolized drug used as reference in the same experiment, suggesting that 23 was degraded quite easily in the assay system. Comparatively, it took about 125 minutes to lose 50% of the newly designed analog 38, indicating a much longer half life (Table 3). This result might be due to the increased steric hindrance at the C-28 pharmacophore of 38, so that the amide bond would be less available to metabolic enzymes. Thus, 38 represents a more stable lead for the development of C-28 modified BA derived HIV-1 entry inhibitors and 3,28-disubstituted bifunctional inhibitors.

TABLE 3
In vitro metabolic stability of compounds 23 and 38. ^{c}

| | |
|---|---|
| | Persentage of Remaining Parent |
| Incubation Time | Compounds (X ± SD) % |

| (min) | Compd. 23 | Compd. 38 |
|---|---|---|
| 0 | 100.0 | 100.0 |
| 5 | 93.4 ± 2.5 | 101.7 ± 4.7 |
| 15 | 70.7 ± 1.5 | 97.6 ± 6.2 |
| 30 | 61.9 ± 10.1 | 81.4 ± 8.6 |
| 60 | 40.1 ± 1.6 | 62.7 ± 4.3 |
| 120 | 7.9 ± 4.2 | 53.8 ± 3.8 |

^{c} Data presented are averages of two separate experiments.

From the bioassay data, we discovered that compound 39 with the C-3 side chain of 2 incorporated into 38, showed very potent anti-HIV-1 activity with an EC₅₀ value of 0.015 μM and TI of 1.4×10³, proving that the presence of a bulky amide moiety near C-28 position does not reduce the antiviral potency of 2. Compound 40, with a 4′,4′-dimethylglutaryl rather than 3′,3′-dimethylsuccinyl C-3 ester side chain, had a higher antiviral EC₅₀ value (0.23 μM), indicating the importance of the positioning of the dimethyl substitution in the C-3 modification of BA. However, compound 38 itself showed a decreased antiviral activity compared with 23. This is likely due to the slightly reduced length of the new C-28 side chain compared with previous 8-aminooctanoic chain (23). Indeed, the BA derivative with 7-aminoheptanoic acid as C-28 side chain showed 16-fold decreased anti-HIV activity compared with 23,¹⁹ confirming the importance of the length of the C-28 side chain.

Compounds 47 and 48, which contain the 3′,3′-dimethylsuccinyl as C-3 side chain and a morpholine ring at the end of C-28 side chain that is separated from the terminal amide bond by a short alkyl spacer (two or three methylenes), exhibited extremely potent anti-HIV-1 replication activity against HIV-1_{IIIB} with EC₅₀ values of 0.007 μM and 0.006 μM, respectively, which are almost 2-fold better than that of 2 and around 10-fold more potent than that of AZT. Their activities are also 2-3 fold more potent than entry inhibitors 3b and 4 in the anti-HIV screening against HIV-1_{NL4-3}. Compound 46, with morpholine directly involved in the amide bond, had similar anti-HIV-1_{IIIB} potency (EC₅₀: 0.011 μM) to that of 2. Compound 45 with methylamine at the end of C-28 showed a slightly decreased antiviral potency (EC₅₀: 0.067 μM) against HIV-1_{IIIB} compared with 2. These results further confirm the importance of the length of the C-28 side chain to the enhanced antiviral potency in 3,28-disubstituted BA analogs. The better potency of 47 and 48 indicates that the activity of 2 can be further increased with proper C-28 substitution. Moreover, because the C-28 side chains in all prior potent HIV inhibitors terminate in a free carboxylic acid or amide, the success of 47 and 48 also demonstrates that other polar groups at the end of this side chain can also increase antiviral potency.

In conclusion, diverse 28,30-disubstituted BA analogs were synthesized in our study. We discovered that a hydrogen bond donor is not tolerated near the C-19 isopropenyl moiety. Otherwise, C-30 substitution did not significantly influence the anti-HIV-1 activity of BA derivatives. Therefore, the C-30 position serves as a good place to incorporate water-solubilizing moieties to increase the hydrophilicity. The resulting analog 21 showed a good solubility as well as equal potency against HIV-1 compared with the previous best anti-entry hit A43-D (4). Using a cyclic secondary amine moiety (piperidine) rather than a primary amine to form the C-28 amide bond significantly increased the metabolic stability of the derivatives in pooled human liver microsome assessment. Subsequent introduction of a second amide bond at the carboxylic terminus of this metabolically stable C-28 side chain and introduction of the 3′,3′-dimethylsuccinyl side chain at the C-3 position resulted in the discovery of 47 and 48, which showed extremely potent antiviral activity, better than that of AZT and slightly better than that of bevirimat (2). They should serve as attractive promising leads for the development of a next generation of BA derived 3,28-disubstituted HIV-1 inhibitors, as clinical trial candidates.

### Experimental Section

Chemistry. The melting points were measured with a Fisher Johns melting apparatus without correction. ¹H NMR spectra were measured on a 300 MHz Varian Gemini 2000 spectrometer using Me₄Si (TMS) as internal standard. The solvent used was CDCl₃ unless otherwise indicated. Mass spectra were measured on Shimadzu LCMS-2010 (ESI-MS). High resolution mass spectra (HRMS) were measured on Shimadzu LCMS-IT-TOF with ESI interface. Elemental analyses were performed by Atlantic Microlab, Inc., Norcross, Ga. Target compounds were analyzed for C, H and gave values within ±0.4% of the theoretical values. Optical rotations were measured with a Jasco Dip-2000 digital polarimeter at 20° C. at the sodium D line. Thin-layer chromatography (TLC) and preparative thin-layer chromatography (PTLC) were performed on Merck precoated silica gel 60 F-254 plates. Flash+™ and CombiFlash systems (Teledyn-Isco) were used as medium pressure column chromatography. Silica gel (200-400 mesh) from Aldrich, Inc., was used for column chromatography. All other chemicals were obtained from Aldrich, Inc.

3-O-Acetyl-betulinic acid (5): A mixture of 1 (2.1 g), pyridine (1.5 mL) and acetic anhydride (Ac₂O, 20 mL) was stirred at room temperature overnight until it became homogenous. The reaction was then poured into ice-cold water (30 mL) and stirred for 20 min. The crude product was filtered off and purified on a silica-gel column to yield 1.98 g (87% yield) of pure 5; white amorphous powder. Mp 289-291° C. MS (ESI−) m/z: 497.38 (M⁻−H) for C₃₂H₅₀O₄. ¹H NMR (300 MHz, CDCl₃): δ 4.74, 4.61 (1H each, s, H-29), 4.47 (1H, dd, J=9.9, 5.9 Hz, H-3), 3.01 (1H, m, H-19), 2.05 (3H, s, OCOCH₃), 1.69 (3H, s, H-30), 0.97, 0.93, 0.86, 0.84, 0.83 (3H each, s, 5×CH₃).

Syntheses of BA-derivatives 6, 7 and 37. Oxalyl chloride solution (2 M in CH₂Cl₂, 10 mL) was added to 5 (1 eq) in CH₂Cl₂ (10 mL) and stirred for 2 h. After concentration under vacuum, the residual mixture was treated with leucine methyl ester (1.6 eq), 8-aminooctanoic acid methyl ester (1.6 eq), or 4-piperidine butyric acid methyl ester (1.6 eq) and triethylamine (Et₃N, 1.2 eq) in CH₂Cl₂. The mixture was stirred at room temperature overnight until no starting material was observed by TLC. The solution was then diluted with CH₂Cl₂ (20 mL) and washed three times with brine and distilled water. The organic layer was dried over anhydrous Na₂SO₄ and concentrated to dryness under reduced pressure. The residue was chromatographed using a silica gel column to yield the pure target compounds.

Methyl N-[3β-Acetoxy-lup-20(29)-en-28-oyl]-leucinate (6): 1.15 g (80.5% yield) starting from 1 g of 5; white amorphous powder. Mp 230-232° C. MS (ESI+) m/z: 626.48 (M⁺+H), 648.47 (M⁺+Na) for C₃₉H₆₃NO₅. ¹H NMR (300 MHz, CDCl₃): δ 5.87 (1H, d, J=8 Hz, —CONH—), 4.72, 4.59 (1H each, s, H-29), 4.64 (1H, m, —NHCH—), 4.49 (1H, t, J=8 Hz, H-3), 3.73 (3H, s, —COOCH₃), 3.05 (1H, m, H-19), 2.10-2.20 (1H, m, H-13), 2.04 (3H, s, OCOCH₃), 1.68 (3H, s, H-30), 1.01 (6H, s, leucine moiety —(CH₃)₂), 0.97 (6H, s, 2×CH₃), 0.89, 0.84, 0.83 (3H each, s, 3×CH₃).

Methyl N-[3β-Acetoxy-lup-20(29)-en-28-oyl]-8-aminooctanoate (7): 643 mg (98% yield) starting from 500 mg of 5; light yellow amorphous powder. Mp 104-105° C. MS (ESI+) m/z: 654.5 (M⁺+H) for C₄₁H₆₇NO₅. ¹H NMR (300 MHz, CDCl₃): δ 5.57 (1H, t, J=6 Hz, —CONH—), 4.73, 4.60 (1H each, s, H-29), 4.45 (1H, m, H-3), 3.67 (3H, s, —COOCH₃), 3.30-3.08 (3H, m, H-19, —CONHCH₂—), 2.50 (1H, m, H-13), 2.31 (2H, t, J=7 Hz, —CH₂COOCH₃), 2.05 (3H, s, OCOCH₃), 1.68 (3H, s, H-30), 0.97, 0.94 (3H each, s, 2×CH₃), 0.85, 0.84, 0.81 (3H each, s, 3×CH₃).

Methyl N-[3β-Acetoxy-lup-20(29)-en-28-oyl]-4-piperidine butanoate (37): 1.02 g (94% yield) starting from 800 mg of 5; white amorphous powder. Mp 195-197° C. MS (ESI+) m/z: 666.5 (M⁺+H) for C₄₂H₆₇NO₅. ¹H NMR (300 MHz, CDCl₃): δ 4.72, 4.57 (1H each, s, H-29), 4.47 (1H, dd, J=11.1, 5.7 Hz, H-3), 3.67 (3H, s, —COOCH₃), 3.67-3.47 (4H, m, 28-CON(CH₂CH₂)₂CH—), 2.99 (1H, m, H-19), 2.31 (2H, t, J=7 Hz, —CH₂COOCH₃), 2.05 (3H, s, OCOCH₃), 1.68 (3H, s, H-30), 0.96 (6H, s, 2×CH₃), 0.94, 0.82, 0.75 (3H each, s, 3×CH₃).

Syntheses of BA-derivatives 8 and 9. A mixture of N-bromosuccinimide (1.1 eq) and 6 or 7 (1 eq) in acetonitrile (ACN, 30 mL) was stirred at room temperature until the starting material was not observed by TLC. The reaction was concentrated to dryness under reduced pressure and chromatographed over silica gel to yield pure target compounds.

Methyl N-[3β-Acetoxy-30-bromo-lup-20(29)-en-28-oyl]-leucinate (8): 297 mg (66% yield) starting from 400 mg of 6; light yellow amorphous powder. Mp 127-129° C. MS (ESI+) m/z: 704.4 (M⁺+H), 706.4 (M⁺+H) for C₃₉H₆₂BrNO₅. ¹H NMR (300 MHz, CDCl₃): δ5.81 (1H, d, J=8 Hz, —CONH—), 5.11, 5.05 (1H each, s, H-29), 4.65 (1H, m, —NHCH—), 4.45 (1H, t, J=8 Hz, H-3), 4.00 (2H, s, H₂-30), 3.72 (3H, s, —COOCH₃), 3.10 (1H, m, H-19), 2.50-2.32 (1H, m, H-13), 2.05 (3H, s, OCOCH₃), 1.02 (6H, s, leucine moiety —(CH₃)₂), 0.99 (6H, s, 2×CH₃), 0.89, 0.86, 0.85 (3H each, s, 3×CH₃).

Methyl N-[3β-Acetoxy-30-bromo-lup-20(29)-en-28-oyl]-8-aminooctanoate (9): 402 mg (72.5% yield) starting from 360 mg of 7; light yellow amorphous powder. Mp 99-101° C. MS (ESI+) m/z: 732.4 (M⁺+H), 734.4 (M⁺+H) for C₄₁H₆₆BrNO₅. ¹H NMR (300 MHz, CDCl₃): δ 5.59 (1H, t, J=6 Hz, —CONH—), 5.13, 5.04 (1H each, s, H-29), 4.47 (1H, t, J=8.1 Hz, H-3), 4.00 (2H, s, H₂-30), 3.67 (3H, s, —COOCH₃), 3.41-3.09 (3H, m, H-19, —CONHCH₂—), 2.46 (1H, m, H-13), 2.31 (2H, t, J=7.5 Hz, —CH₂COOCH₃), 2.04 (3H, s, OCOCH₃), 0.97, 0.93 (3H each, s, 2×CH₃), 0.89, 0.84, 0.83 (3H each, s, 3×CH₃).

Syntheses of BA-derivatives 10-20. NaH (60% in mineral oil) was washed three times with hexane. A solution of appropriate nucleophilic compound (8 eq) and NaH (10 eq) in anhydrous THF (1.5 mL) was stirred under dry nitrogen at room temperature for 30 min. The 30-bromo BA derivative 8 or 9 (1 eq) was then added into the system. The reaction was heated using microwave (Biotage) at 120° C. for 30 min. After cooling to room temperature, 1 mL MeOH—H₂O was added into the mixtures and stirred to transform the intermediate esters to carboxylic acids by saponification. The reaction was neutralized with 10% HCl and dried under vacuum and reconstituted with EtOAc. The organic layer was washed with brine and dried over anhydrous Na₂SO₄ and concentrated to dryness under reduced pressure. The residue was chromatographed using a silica gel column to yield the pure target compounds.

N-[3β-Hydroxy-30-ethoxy-lup-20(29)-en-28-oyl]-leucine (10): 22 mg (59% yield) starting from 40 mg of 8; white amorphous powder. Mp 128-130° C. MS (ESI−) m/z: 612.4 (M⁻−H) for C₃₈H₆₃NO₅. ¹H NMR (300 MHz, CDCl₃): δ 5.88 (1H, d, J=8 Hz, —CONH—), 4.93, 4.92 (2H, br s, H-29), 4.63-4.58 (1H, m, —NHCH—), 3.90 (2H, s, H₂-30), 3.47 (2H, m, 30-OCH₂CH₃), 3.18 (1H, dd, J=11.1, 5.4 Hz, H-3), 2.99 (1H, m, H-19), 2.50-2.32 (1H, m, H-13), 1.00 (9H, br s, 30-OCH₂CH₃, leucine moiety —(CH₃)₂), 0.96 (6H, s, 2×CH₃), 0.89, 0.86, 0.85 (3H each, s, 3×CH₃).

N-[3β-Hydroxy-30-propoxy-lup-20(29)-en-28-oyl]-leucine (11): 23 mg (60% yield) starting from 40 mg of 8; white amorphous powder. Mp 116-117° C. MS (ESI−) m/z: 626.5 (M⁻−H) for C₃₉H₆₅NO₅. ¹H NMR (300 MHz, CDCl₃): δ 6.13 (1H, br s, —CONH—), 4.91, 4.90 (2H, br s, H-29), 4.52 (1H, m, —NHCH—), 3.90 (2H, s, H₂-30), 3.36 (2H, t, J=6.9 Hz, 30-OCH₂CH₂CH₃), 3.18 (1H, dd, J=11.1, 5.4 Hz, H-3), 2.99 (1H, m, H-19), 0.96, 0.94, 0.92, 0.89 (15H, m, 30-O(CH₂)₂CH₃, leucine moiety —(CH₃)₂, CH₃-23, 24), 0.82, 0.81, 0.79 (3H each, s, 3×CH₃).

N-[3β-Hydroxy-30-butoxy-lup-20(29)-en-28-oyl]-leucine (12): 10 mg (37% yield) starting from 30 mg of 8; yellow amorphous powder. Mp 104-105° C. MS (ESI−) m/z: 640.2 (M⁻−H) for C₄₀H₆₇NO₅. ¹H NMR (300 MHz, CDCl₃): δ 6.01 (1H, br s, —CONH—), 4.90, 4.88 (2H, br s, H-29), 4.58 (1H, m, —NHCH—), 3.89 (2H, s, H₂-30), 3.37 (2H, m, 30-OCH₂(CH₂)₂CH₃), 3.17 (1H, m, H-3), 3.01 (1H, m, H-19), 0.99 (9H, br s, 30-O(CH₂)₃CH₃, leucine moiety —(CH₃)₂), 0.96 (6H, s, 2×CH₃), 0.86, 0.84, 0.81 (3H each, s, 3×CH₃).

N-[3β-Hydroxy-30-phenethoxy-lup-20(29)-en-28-oyl]-leucine (13): 37 mg (77% yield) starting from 50 mg of 8; light yellow amorphous powder. Mp 155-157° C. MS (ESI−) m/z: 688.4 (M⁻−H). ¹H NMR (300 MHz, CDCl₃): δ 7.68-7.62 (2H, m, H ar-3′), 7.28-7.20 (3H, m, H ar-2′,4′), 5.97 (1H, br s, —CONH—), 4.91, 4.90 (2H, br s, H-29), 4.44 (1H, m, —NHCH—), 3.93 (2H, s, H₂-30), 3.64 (2H, t, J=7.2 Hz, 30-OCH₂CH₂Ph), 3.17 (1H, dd, J=11.1, 5.4 Hz, H-3), 2.91 (1H, m, H-19), 2.57 (2H, m, 30-OCH₂CH₂Ph), 0.95 (12H, s, leucine moiety —(CH₃)₂, CH₃-23, 24), 0.89, 0.78, 0.74 (3H each, s, 3×CH₃). Anal. (C₄₄H₆₇O₅N.2H₂O) C, H, O.

N-[3β-Hydroxy-30-(4′-methoxyphenethoxy)-lup-20(29)-en-28-oyl]-leucine (14): 46 mg (64% yield) starting from 70 mg of 8; light yellow amorphous powder. Mp 128-129° C. MS (ESI−) m/z: 718.5 (M⁻−H). ¹H NMR (300 MHz, CDCl₃): δ 7.27, 7.16-7.13, 6.85-6.82 (5H, m, H ar-2′,3′,4′), 5.97 (1H, br s, —CONH—), 4.91, 4.89 (H each, br s, H-29), 4.48 (1H, m, —NHCH—), 3.93 (2H, s, H₂-30), 3.79 (3H, s, ar-OCH₃), 3.60 (2H, t, J=7.2 Hz, 30-OCH₂CH₂Ph(p-OCH₃)), 3.17 (1H, dd, J=11.1, 5.4 Hz, H-3), 2.85 (1H, t, J=7.5 Hz, H-19), 2.39 (3H, m, 30-OCH₂CH₂Ph(p-OCH₃), H-13), 0.95, 0.93, 0.90 (15H, s, leucine moiety —(CH₃)₂, 3×CH₃), 0.79, 0.75 (3H each, s, 2×CH₃). Anal. (C₄₅H₆₉O₆N.4½H₂O) C, H, O.

N-[3β-Hydroxy-30-(4% fluorophenethoxy)-lup-20(29)-en-28-oyl]-leucine (15): 24 mg (40% yield) starting from 60 mg of 8; light yellow amorphous powder. Mp 102-104° C. MS (ESI−) m/z: 706.4 (M⁻−H). ¹H NMR (300 MHz, CDCl₃): δ 7.16-6.80 (5H, m, H ar-2′,3′,4′), 5.96 (1H, br s, —CONH—), 4.90, 4.89 (2H, br s, H-29), 4.48 (1H, m, —NHCH—), 3.92 (2H, s, H₂-30), 3.61 (2H, t, J=7.2 Hz, 30-OCH₂CH₂Ph(p-F)), 3.17 (1H, m, H-3), 2.87 (1H, t, J=7.5 Hz, H-19), 2.36-2.10 (3H, m, 30-OCH₂CH₂Ph(p-F), H-13), 0.95, 0.88 (15H, s, leucine moiety —(CH₃)₂, 3×CH₃), 0.75, 0.73 (3H each, s, 2×CH₃). Anal. (C₄₄H₆₆O₅NF.3½H₂O) C, H, O.

N-[3β-Hydroxy-30-(4′-bromophenethoxy)-lup-20(29)-en-28-oyl]-leucine (16): 18 mg (41% yield) starting from 40 mg of 8; light yellow amorphous powder. Mp 127-129° C. MS (ESI−) m/z: 706.4 (M⁻−H). ¹H NMR (300 MHz, CDCl₃): δ 7.56-7.28 (5H, m, H ar-2′,3′,4′), 5.96 (1H, br s, —CONH—), 4.91, 4.90 (2H, br s, H-29), 4.48 (1H, m, —NHCH—), 3.91 (2H, s, H₂-30), 3.60 (2H, t, J=7.0 Hz, 30-OCH₂CH₂Ph(p-Br)), 3.17 (1H, dd, J=11.0, 5.6 Hz, H-3), 2.89 (1H, t, J=7.5 Hz, H-19), 2.39 (1H, m, 30-OCH₂CH₂Ph(p-Br)), 0.96 (12H, s, leucine moiety —(CH₃)₂, 2×CH₃), 0.82, 0.79, 0.75 (3H each, s, 3×CH₃). Anal. (C₄₄H₆₆O₅NBr.2H₂O) C, H, O.

N-[3β-Hydroxy-30-(4′-chlorophenethoxy)-lup-20(29)-en-28-oyl]-leucine (17): 16 mg (38% yield) starting from 40 mg of 8; light yellow amorphous powder. Mp 119-121° C. MS (ESI−) m/z: 706.4 (M⁻−H). ¹H NMR (300 MHz, CDCl₃): δ 7.18-6.87 (5H, m, H ar-2′,3′,4′), 5.96 (1H, br s, —CONH—), 4.91, 4.90 (2H, br s, H-29), 4.48 (1H, m, —NHCH—), 3.91 (2H, s, H₂-30), 3.62 (2H, t, J=6.8 Hz, 30-OCH₂CH₂Ph(p-Cl)), 3.17 (1H, dd, J=11.0, 5.6 Hz, H-3), 2.87 (1H, t, J=7.5 Hz, H-19), 2.36-2.06 (3H, m, 30-OCH₂CH₂Ph(p-Cl), H-13), 0.96 (15H, s, leucine moiety —(CH₃)₂, 3×CH₃), 0.81, 0.76 (3H each, s, 2×CH₃). Anal. (C₄₄H₆₆O₅NCl.H₂O) C, H, O.

N-[3β-Hydroxy-30-morpholino-lup-20(29)-en-28-oyl]-leucine (18): 22 mg (41% yield) starting from 60 mg of 8; white amorphous powder. Mp 98-100° C. MS (ESI−) m/z: 653.5 (M⁻−H). ¹H NMR (300 MHz, CDCl₃): δ 5.61 (1H, d, J=6 Hz, —CONH—), 4.92, 4.90 (H each, s, H-29), 4.63-4.58 (1H, m, —NHCH—), 3.72 (4H, m, 30-N(CH₂CH₂)₂O), 3.17 (1H, dd, J=11.1, 5.4 Hz, H-3), 3.00 (3H, m, H-19, H₂-30), 2.53 (4H, m, 30-N(CH₂CH₂)₂O), 2.42 (1H, m, H-13), 0.96 (6H, s, leucine moiety —(CH₃)₂), 0.92 (6H, s, 2×CH₃), 0.86, 0.81, 0.75 (3H each, s, 3×CH₃). Anal. (C₄₀H₆₆O₅N₂.2H₂O) C, H, O.

N-[3β-Hydroxy-30-(2′-morpholinoethoxy)-lup-20(29)-en-28-oyl]-leucine (19): 26 mg (56% yield) starting from 50 mg of 8; white amorphous powder. Mp 89-91° C. MS (ESI−) m/z: 697.4 (M⁻−H). ¹H NMR (300 MHz, CDCl₃): δ 5.61 (1H, d, J=8 Hz, —CONH—), 4.92, 4.90 (H each, s, H-29), 4.59 (1H, m, —NHCH—), 3.94 (2H, s, H₂-30), 3.72 (4H, m, −N(CH₂CH₂)₂O), 3.58 (2H, t, J=5.7 Hz, 30-OCH₂CH₂-morpholine), 3.18 (1H, dd, J=11.4, 4.6 Hz, H-3), 3.01 (1H, m, H-19), 2.60 (2H, t, J=5.4 Hz, 30-OCH₂CH₂-morpholine), 2.53 (4H, m, —N(CH₂CH₂)₂O), 1.00 (6H, s, leucine moiety —(CH₃)₂), 0.96 (6H, s, 2×CH₃), 0.89, 0.85, 0.80 (3H each, s, 3×CH₃). Anal. (C₄₂H₇₀O₆N₂.H₂O) C, H, O.

N-[3β-Hydroxy-30-(2′-morpholinoethoxy)-lup-20(29)-en-28-oyl]-8-aminooctanoic acid (20): 51 mg (64% yield) starting from 80 mg of 9; white amorphous powder. Mp 111-112° C. MS (ESI−) m/z: 725.5 (M⁻−H) for C₄₄H₇₄N₂O₆. ¹H NMR (300 MHz, CDCl₃): δ 5.61 (1H, d, J=8 Hz, —CONH—), 4.91, 4.90 (H each, s, H-29), 3.94 (2H, s, H₂-30), 3.72 (4H, m, —N(CH₂CH₂)₂O), 3.58 (2H, t, J=5.7 Hz, 30-OCH₂CH₂-morpholine), 3.18 (3H, m, —CONHCH₂—, H-3), 3.01 (1H, m, H-19), 2.60 (2H, t, J=5.4 Hz, 30-OCH₂CH₂-morpholine), 2.53 (4H, m, —N(CH₂CH₂)₂O), 2.28 (2H, t, J=7.5 Hz, —CH₂COOH), 0.96 (6H, s, 2×CH₃), 0.92, 0.81, 0.75 (3H each, s, 3×CH₃).

Syntheses of BA-derivatives 26 and 27. A solution of 30-bromo BA derivative 8 or 9 (1 eq), silver acetate (AgOAc, 2 eq) and tetrabutylammonium bromide (Bu₄NBr, 0.2 eq) in acetonitrile (1.5 mL) was heated using microwave at 100° C. for 25 min. The precipitant was filtered and the solution was concentrated to dryness under vacuum. The residue was chromatographed over a silica-gel column to yield the pure diacetoxy intermediates 26 and 27.

Methyl N-[3β,30-diacetoxy-lup-20(29)-en-28-oyl]-leucinate (26): 80 mg (68% yield) starting from 120 mg of 117; off-white amorphous powder. Mp 201-203° C. MS (ESI+) m/z: 684.5 (M⁺+H) for C₄₁H₆₅NO₇. ¹H NMR (300 MHz, CDCl₃): δ 5.69 (1H, br s, —CONH—), 4.97, 4.94 (2H, d, J=9, H-29), 4.58-4.52 (3H, m, —NHCH—, H₂-30), 4.45 (1H, t, J=8 Hz, H-3), 3.72 (3H, s, —COOCH₃), 3.10 (1H, m, H-19), 2.50-2.32 (1H, m, H-13), 2.08 (6H, s, 2×OCOCH₃), 1.05 (6H, s, leucine moiety —(CH₃)₂), 0.96 (6H, s, 2×CH₃), 0.89, 0.82, 0.81 (3H each, s, 3×CH₃).

Methyl N-[3β,30-diacetoxy-lup-20(29)-en-28-oyl]-8-aminooctanoate (27): 77.8 mg (69.5% yield) starting from 80 mg of 118; white amorphous powder. Mp 167-169° C. MS (ESI+) m/z: 712.5 (M⁺+H) for C₄₃H₆₉NO₇. ¹H NMR (300 MHz, CDCl₃): δ 5.60 (1H, t, J=4.6 Hz, —CONH—), 4.94, 4.90 (1H each, s, H-29), 4.56 (2H, s, H₂-30), 4.45 (1H, t, J=7 Hz, H-3), 3.66 (3H, s, —COOCH₃), 3.41-3.09 (3H, m, H-19, —CONHCH₂—), 2.46 (1H, m, H-13), 2.31 (2H, t, J=7.5 Hz, —CH₂COOCH₃), 2.05 (6H, s, 2×OCOCH₃), 0.97, 0.96, 0.85, 0.81, 0.80 (3H each, s, 5×CH₃).

Syntheses of 1-derivatives 22-25, 28-29, 36 and 38. To a solution of the appropriate ester intermediates 6-9, 26-27, 35 and 37 (1 eq) in MeOH (8 mL) and THF (4 mL) was added 2 N NaOH (4 mL). The mixture was stirred overnight, and then neutralized with 20% HCl. The solution was dried under vacuum and reconstituted with EtOAc. The organic layer was washed with brine and dried over anhydrous Na₂SO₄ and concentrated to dryness under reduced pressure. The residue was chromatographed using a silica gel column to yield the pure target compounds.

N-[3β-Hydroxy-lup-20(29)-en-28-oyl]-leucine (22): 27 mg (100% yield) starting from 30 mg of 6; white amorphous powder. Mp 243-244° C. MS (ESI−) m/z: 568.42 (M⁻−H) for C₃₆H₅₉NO₄. ¹H NMR (300 MHz, CDCl₃): δ 5.86 (1H, d, J=8 Hz, —CONH—), 5.11, 5.02 (1H each, s, H-29), 4.65 (1H, m, —NHCH—), 3.17 (1H, dd, J=9.7, 5.4 Hz, H-3), 3.10-3.03 (1H, m, H-19), 1.68 (3H, s, H-30), 1.00 (6H, s, leucine moiety —(CH₃)₂), 0.96 (6H, s, 2×CH₃), 0.83, 0.80, 0.79 (3H each, s, 3×CH₃). [α]²⁵_{D} −17.2° (c=1.40, CHCl₃).

N-[3β-Hydroxy-lup-20(29)-en-28-oyl]-8-aminooctanoic acid (23): 37 mg (100% yield) starting from 40 mg of 7; white amorphous powder. Mp 110-112° C. MS (ESI−) m/z: 596.5 (M⁻−H) for C₃₈H₆₃NO₄. ¹H NMR (300 MHz, CDCl₃): δ5.60 (1H, br s, —CONH—), 4.73, 4.60 (1H each, s, H-29), 3.21-3.09 (4H, m, H-3, H-19, —CONHCH₂—), 2.31 (2H, t, J=6.9 Hz, —CH₂COOH), 2.10-2.20 (1H, m, H-13), 1.68 (3H, s, H-30), 0.97 (6H, s, 2×CH₃), 0.85, 0.79, 0.75 (3H each, s, 3×CH₃). [α]²⁵_{D} −3.6° (c=0.19, CHCl₃). [α]²⁵_{D} −8.48° (c=0.20, MeOH).

N-[3β-Hydroxy-30-bromo-lup-20(29)-en-28-oyl]-leucine (24): 102 mg (100% yield) starting from 110 mg of 8; white amorphous powder. Mp 102-104° C. MS (ESI−) m/z: 646.41, 648.39 (M⁻−H) for C₃₆H₅₈BrNO₄. ¹H NMR (300 MHz, CDCl₃): δ 5.86 (1H, d, J=8 Hz, —CONH—), 5.11, 5.02 (1H each, s, H-29), 4.65 (1H, m, —NHCH—), 3.90 (2H, s, H₂-30), 3.17 (1H, dd, J=9.7, 5.4 Hz, H-3), 3.10-3.03 (1H, m, H-19), 1.00 (6H, s, leucine moiety —(CH₃)₂), 0.96 (6H, s, 2×CH₃), 0.83, 0.80, 0.79 (3H each, s, 3×CH₃). [α]²⁵_{D} −10.5° (c=0.15, MeOH).

N-[3β-Hydroxy-30-bromo-lup-20(29)-en-28-oyl]-8-aminooctanoic acid (25): 44 mg (95% yield) starting from 50 mg of 9; white amorphous powder. Mp 119-122° C. MS (ESI−) m/z: 674.4 (M⁻−H) for C₃₈H₆₂BrNO₄. ¹H NMR (300 MHz, CDCl₃): δ5.60 (1H, br s, —CONH—), 5.13, 5.04 (1H each, s, H-29), 4.00 (2H, s, H₂-30), 3.21-3.09 (4H, m, H-3, H-19, —CONHCH₂—), 2.34 (2H, m, —CH₂COOH), 0.96 (6H, s, 2×CH₃), 0.92, 0.82, 0.81 (3H each, s, 3×CH₃). [α]²⁵_{D} −16.5° (c=0.22, MeOH).

N-[3β,30-Dihydroxy-lup-20(29)-en-28-oyl]-leucine (28): 24 mg (98% yield) starting from 30 mg of 26; white amorphous powder. Mp 145-148° C. MS (ESI−) m/z: 584.5 (M⁻−H) for C₃₆H₅₉NO₅. ¹H NMR (300 MHz, CDCl₃): δ 5.89 (1H, br s, —CONH—), 4.91, 4.90 (1H each, s, H-29), 4.68 (1H, m, —NHCH—), 4.12 (2H, s, H₂-30), 3.17 (1H, dd, J=11.2, 5.6 Hz, H-3), 3.01 (1H, m, H-19), 2.34 (1H, m, H-13), 1.10 (6H, s, leucine moiety —(CH₃)₂), 0.99 (6H, s, 2×CH₃), 0.86, 0.83, 0.80 (3H each, s, 3×CH₃). [α]²⁵_{D} −39.3° (c=0.35, MeOH).

N-[3β,30-Dihydroxy-lup-20(29)-en-28-oyl]-8-aminooctanoic acid (29): 50 mg (80% yield) starting from 58 mg of 27; white amorphous powder. Mp 135-137° C. MS (ESI−) m/z: 612.5 (M⁻−H) for C₃₈H₆₃NO₅. ¹H NMR (300 MHz, CDCl₃): δ5.61 (1H, br s, —CONH—), 4.94, 4.90 (1H each, s, H-29), 4.12 (2H, s, H₂-30), 3.25-3.15 (3H, m, H-3, —CONHCH₂—), 3.01 (1H, m, H-19), 2.34 (2H, t, J=7.6 Hz, —CH₂COOH), 2.06 (1H, m, H-13), 0.97, 0.96 (3H each, s, 2×CH₃), 0.92, 0.82, 0.75 (3H each, s, 3×CH₃). [α]²⁵_{D} −143.3° (c=0.10, MeOH).

N′-[3β-(3′,3′-Dimethylsuccinyl)-lup-20(29)-en-28-oyl]-leucine (36): 24 mg (98%) starting from 25 mg of 35; white amorphous powder. Mp 248-250° C. MS (ESI−) m/z: 695.5 (M⁻−H) for C₄₂H₆₈N₂O₆. ¹H NMR (300 MHz, CDCl₃): δ 5.87 (1H, d, J=7.6 Hz, —CONH—), 4.72, 4.58 (1H each, s, H-29), 4.64 (1H, m, —NHCH—), 3.59 (1H, m, H-3), 2.99 (1H, m, H-19), 2.64-2.42 (2H, m, H-2′), 1.68 (3H, s, H-30), 1.30, 1.26 (3H each, s, 2×CH₃-3′), 1.00 (6H, s, leucine moiety —(CH₃)₂), 0.96 (6H, s, 2×CH₃), 0.89, 0.86, 0.85 (3H each, s, 3×CH₃). [α]²⁵_{D}−16.1° (c=0.28, MeOH).

N-[3β-Hydroxy-lup-20(29)-en-28-oyl]-4-piperidine butyric acid (38): 190 mg (100%) starting from 200 mg of 37, white amorphous powder. Mp 145-146° C. MS (ESI−) m/z: 608.4 (M⁻−H) for C₃₉H₆₃NO₄. ¹H NMR (300 MHz, CDCl₃): δ 4.72, 4.57 (1H each, s, H-29), 3.67-3.47 (4H, m, 28-CON(CH₂CH₂)₂CH—), 3.19 (1H, m, H-3), 2.99 (1H, m, H-19), 2.31 (2H, t, J=8.4 Hz, —CH₂COOH), 1.68 (3H, s, H-30), 0.96 (6H, s, 2×CH₃), 0.94, 0.82, 0.81 (3H each, s, 3×CH₃). [α]²⁵_{D} −22.7° (c=0.33, MeOH).

Synthesis of BA-derivatives 21 and 41-44. A solution of 20 or 38 (1 eq), EDCI (2 eq), N-Hydroxybenzotriazole (HOBt, 1 eq), Et₃N (0.05 mL) and the appropriate amine (2 eq) in anhydrous CH₂Cl₂ (8 mL) was stirred at room temperature overnight until the starting material was not observed by TLC. The solution was diluted with CH₂Cl₂ (20 mL) and washed three times with brine and distilled water. The organic layer was dried over anhydrous Na₂SO₄ and concentrated to dryness under reduced pressure. The residue was chromatographed using a silica gel column to yield pure target compounds.

β-Hydroxy-30-(2′-morpholinoethoxy)-lup-20(29)-en-28-oyl]-8-aminooctanoyl]-aminomethane (21): 21 mg (69% yield) starting from 30 mg of 20; white amorphous powder. Mp 106-107° C. MS (ESI+) m/z: 740.5 (M⁺+H) for C₄₅H₇₇N₃O₅. ¹H NMR (300 MHz, CDCl₃): δ 5.61 (2H, m, 2×-CONH—), 4.92, 4.90 (H each, s, H-29), 3.94 (2H, s, H₂-30), 3.72 (4H, m, —N(CH₂CH₂)₂O), 3.58 (2H, t, J=5.7 Hz, 30-OCH₂CH₂-morpholine), 3.28-3.14 (3H, m, —CONHCH₂—, H-3), 3.01 (1H, m, H-19), 2.81 (3H, d, J=4.8 Hz, —CONHCH₃), 2.60 (2H, t, J=5.4 Hz, 30-OCH₂CH₂-morpholine), 2.53 (4H, m, —N(CH₂CH₂)₂O), 2.16 (2H, t, J=7.5 Hz, —CH₂CONHCH₃), 0.96 (6H, s, 2×CH₃), 0.92, 0.81, 0.75 (3H each, s, 3×CH₃).

N′-[N-[3β-Hydroxy-lup-20(29)-en-28-oyl]-4-piperidine-butanoyl]-aminomethane (41): 46 mg (100% yield) starting from 50 mg of 38, off-white amorphous powder. Mp 202-204° C. MS (ESI+) m/z: 623.5 (M⁺+H) for C₄₀H₆₆N₂O₃. ¹H NMR (300 MHz, CDCl₃): δ 5.44 (1H, br s, —CONHCH₃), 4.69, 4.54 (1H each, s, H-29), 3.58-3.54 (4H, m, 28-CON(CH₂CH₂)₂CH—), 3.16 (1H, m, H-3), 2.95 (1H, m, H-19), 2.78 (3H, d, J=4.8 Hz, —CONHCH₃), 2.16 (2H, t, J=7.5 Hz, —CH₂CONHCH₃), 1.66 (3H, s, H-30), 0.93 (6H, s, 2×CH₃), 0.92, 0.90, 0.79 (3H each, s, 3×CH₃). [α]²⁵_{D} −10.7° (c=0.19, MeOH).

β-Hydroxy-lup-20(29)-en-28-oyl]-4-piperidine-butanoyl]-morpholine (42): 53 mg (87% yield) starting from 50 mg of 38, white amorphous powder. Mp 132-133° C. MS (ESI+) m/z: 679.5 (M⁺+H) for C₄₃H₇₀N₂O₄. ¹H NMR (300 MHz, CDCl₃): δ 4.69, 4.54 (1H each, s, H-29), 3.66-3.60 (8H, m, 28-CON(CH₂CH₂)₂CH—, —CON(CH₂CH₂)₂O), 3.42 (4H, m, —CON(CH₂CH₂)₂O), 3.14 (1H, m, H-3), 2.95-2.60 (1H, m, H-19), 2.27 (2H, t, J=9.2 Hz, —CH₂CON(CH₂CH₂)₂O), 1.65 (3H, s, H-30), 0.93 (3H each, s, 2×CH₃), 0.91, 0.83, 0.79 (3H each, s, 3×CH₃). [α]²⁵_{D} −20.0° (c=0.31, MeOH).

N′-[N-[3β-Hydroxy-lup-20(29)-en-28-oyl]-4-piperidine-butanoyl]-2-aminoethylmorpholine (43): 54 mg (91% yield) starting from 55 mg of 38, white amorphous powder. Mp 114-116° C. MS (ESI+) m/z: 722.6 (M⁺+H), 744.5 (M⁺+Na) for C₄₅H₇₅N₃O₄. ¹H NMR (300 MHz, CDCl₃): δ 6.79 (1H, br, s, —CONHCH₂—), 4.68, 4.53 (1H each, s, H-29), 3.69-3.67 (8H, m, 28-CON(CH₂CH₂)₂CH—, —CH₂N(CH₂CH₂)₂O), 3.31 (2H, m, —CONHCH₂—), 3.17 (1H, m, H-3), 2.95-2.60 (1H, m, H-19), 2.43 (6H, m, —CH₂N(CH₂CH₂)₂O), 2.17 (2H, t, J=7.5 Hz, —CH₂CONHCH₂—), 1.63 (3H, s, H-30), 0.93 (3H each, s, 2×CH₃), 0.90, 0.79, 0.72 (3H each, s, 3×CH₃). [α]²⁵_{D} −10.6° (c=0.15, MeOH).

N′-[N-[3β-Hydroxy-lup-20(29)-en-28-oyl]-4-piperidine-butanoyl]-3-aminopropylmorpholine (44): 54 mg (90% yield) starting from 50 mg of 38, white amorphous powder. Mp 122-124° C. MS (ESI+) m/z: 736.6 (M⁺+H) for C₄₆H₇₇N₃O₄. ¹H NMR (300 MHz, CDCl₃): δ 5.93 (1H, br, s, —CONHCH₂—), 3.15 (1H, m, H-3), 2.95-2.60 (1H, m, H-19), 2.47-2.41 (6H, m, —CH₂N(CH₂CH₂)₂O), 2.16 (2H, t, J=7.5 Hz, —CH₂CONHCH₂—), 1.65 (3H, s, H-30), 0.93 (3H each, s, 2×CH₃), 0.91, 0.79, 0.72 (3H each, s, 3×CH₃). [α]²⁵_{D} −7.5° (c=0.18, MeOH).

3-Deoxy-betulinic acid (31): To a solution of 1 (2 g, 1 eq) in DMF was added pyridium dichromate (PDC, 2 eq). The mixture was stirred at room temperature for 2 h. The reaction mixture was diluted with EtOAc (30 mL) and the precipitate was filtered through a short pack of Florisil. The solution was washed with 20% HCl and distilled water. The organic layer was dried over anhydrous Na₂SO₄ and concentrated to dryness under vacuum. The residue was chromatographed using a silica gel column to yield 1.68 g (87%) of pure 31, white powder. Mp 246-248° C. MS (ESI−) m/z: 453.3 (M⁻−H) for C₃₀H₄₆O₃. ¹H NMR (300 MHz, CDCl₃): δ4.72, 4.58 (1H each, s, H-29), 3.09 (1H, m, H-19), 2.41-2.26 (2H, m, H-2), 1.69 (3H, s, H-30), 0.98, 0.97, 0.96, 0.92, 0.89 (3H each, s, 5×CH₃).

Methyl N-[3-Deoxy-lup-20(29)-en-28-oyl]-leucinate (32): A solution of 31 (500 mg, 1 eq), DMAP (0.6 eq) and EDCI (1.6 eq) in CH₂Cl₂ was stirred at 0° C. for 30 min. Leucine methyl ester (1.6 eq) and Et₃N (1 eq) was then added into the system and stirred at room temperature overnight. The reaction was diluted with CH₂Cl₂ (20 mL) and washed with brine. The organic layer was then dried over anhydrous Na₂SO₄ and concentrated to dryness under vacuum. The residue was chromatographed using a silica gel column to yield 372 mg (58%) of pure 32, white amorphous powder. Mp 191-193° C. MS (ESI+) m/z: 582.5 (M⁺+H) for C₃₇H₅₉NO₄. ¹H NMR (300 MHz, CDCl₃): δ 5.87 (1H, d, J=8.4 Hz, —CONH—), 4.70, 4.59 (1H each, s, H-29), 4.64 (1H, m, —NHCH—), 3.73 (3H, s, —COOCH₃), 3.05 (1H, m, H-19), 2.41-2.26 (2H, m, H-2), 1.68 (3H, s, H-30), 1.06, 1.02 (3H each, s, leucine moiety —(CH₃)₂), 0.98 (3H, s, CH₃), 0.96 (6H, s, 2×CH₃), 0.92, 0.89 (3H each, s, 2×CH₃).

Methyl N-[3-Oxime-lup-20(29)-en-28-oyl]-leucinate (33): A solution of 32 (230 mg, 1 eq), and hydroxylamine hydrochloride (4 eq) in pyridine (10 mL) was heated at 50° C. for 2 h. After cooling to room temperature, the reaction mixture was diluted with CH₂Cl₂ and washed three times by 20% HCl and brine. The organic layer was then dried over anhydrous Na₂SO₄ and concentrated to dryness under vacuum. The residue was chromatographed using a silica gel column to yield 235 mg (90%) of pure 33, white amorphous powder. Mp 213-215° C. MS (ESI+) m/z: 582.5 (M⁺+H) for C₃₇H₅₉NO₄. ¹H NMR (300 MHz, CDCl₃): δ 5.87 (1H, d, J=8.4 Hz, —CONH—), 4.70, 4.59 (1H each, s, H-29), 4.64 (1H, m, —NHCH—), 3.73 (3H, s, —COOCH₃), 3.05 (1H, m, H-19), 2.20-2.15 (2H, m, H-2), 1.67 (3H, s, H-30), 1.05, 1.02 (3H each, s, leucine moiety —(CH₃)₂), 0.98 (3H, s, CH₃), 0.96 (6H, s, 2×CH₃), 0.94, 0.92 (3H each, s, 2×CH₃).

Methyl N-[3β-Amino-lup-20(29)-en-28-oyl]-leucinate (34): To a solution of 33 (100 mg, 1 eq) and ammonium acetate (15 eq) in MeOH was added sodium cyanoborohydride (NaCNBH₃, 20 eq) under nitrogen atmosphere. The reaction was cooled to 0° C., and 15% aqueous titanium trichloride (TiCl₃, 3 eq) was added dropwise over 45 min. The mixture was stirred at room temperature overnight, and then treated with 2 N NaOH until pH=10. The solution was dried under vacuum and the resided aqueous solution was extracted with CH₂Cl₂ and washed with distilled water until pH=7. The organic layer was then dried over anhydrous Na₂SO₄ and concentrated to dryness under vacuum. The residue was chromatographed using a silica gel column to yield 80 mg (82%) of pure 34, white amorphous powder. Mp 135-137° C. MS (ESI+) m/z: 582.5 (M⁺+H) for C₃₇H₅₉NO₄. ¹H NMR (300 MHz, CDCl₃): δ 5.86 (1H, d, J=7 Hz, —CONH—), 4.72, 4.58 (1H each, s, H-29), 4.64 (1H, m, —NHCH—), 3.73 (3H, s, —COOCH₃), 3.05 (1H, m, H-19), 2.44 (1H, m, H-3), 2.10-1.90 (1H, m, H-13), 1.68 (3H, s, H-30), 0.97 (9H, s, CH₃-23, leucine moiety-(CH₃)₂), 0.96, 0.94, 0.93, 0.92 (3H each, s, 4×CH₃).

Synthesis of BA-derivatives 30, 35, 39-40, 45-48. A solution of the appropriate BA analog (1 eq), DMAP (1.5 eq) and the appropriate acid anhydride (5 eq) in anhydrous pyridine (1.5 mL) was stirred at 160° C. for 2 h using microwave (Biotage). The reaction mixture was diluted with EtOAc (15 mL) and washed three times with 20% HCl solution and distilled water. The organic layer was dried over anhydrous Na₂SO₄ and concentrated to dryness under reduced pressure. The residue was chromatographed using a silica gel column to yield pure target compounds.

N-[3β-O-(3′,3′-Dimethylsuccinyl)-30-bromo-lup-20(29)-en-28-oyl]-leucine (30): 20 mg (32% yield) starting from 50 mg of 24; light yellow amorphous powder. Mp 105-107° C. MS (ESI−) m/z: 774.5 (M⁻−H) for C₄₂H₆₆BrNO₇. ¹H NMR (300 MHz, CDCl₃): δ 5.86 (1H, d, J=8 Hz, —CONH—), 5.11, 5.02 (1H each, s, H-29), 4.65 (1H, m, —NHCH—), 4.54 (1H, dd, J=11.2, 5.7 Hz, H-3), 3.90 (2H, s, H₂-30), 2.99 (1H, m, H-19), 2.64-2.42 (2H, m, H-2′), 1.30, 1.26 (3H each, s, 2×CH₃-3′), 1.00 (6H, s, leucine moiety —(CH₃)₂), 0.96 (6H, s, 2×CH₃), 0.87, 0.86, 0.81 (3H each, s, 3×CH₃).

Methyl N′-[3β-N-(3′,3′-Dimethylsuccinyl)-lup-20(29)-en-28-oyl]-leucinate (35): 37 mg (38% yield) starting from 80 mg of 34; white amorphous powder. Mp 187-189° C. MS (ESI+) m/z: 711.5 (M⁺+H), 733.4 (M⁺+Na) for C₄₃H₇₀N₂O₆. ¹H NMR (300 MHz, CDCl₃): δ 5.69 (1H, br s, —CONH—), 4.70, 4.58 (1H each, s, H-29), 4.62 (1H, m, —NHCH—), 3.73 (3H, s, —COOCH₃), 3.59 (1H, m, H-3), 2.99 (1H, m, H-19), 2.64-2.42 (2H, m, H-2′), 1.68 (3H, s, H-30), 1.30, 1.26 (3H each, s, 2×CH₃-3′), 1.09 (6H, s, leucine moiety —(CH₃)₂), 0.97 (6H, s, 2×CH₃), 0.92, 0.82, 0.80 (3H each, s, 3×CH₃).

N-[3β-O-(3′,3′-Dimethylsuccinyl)-lup-20(29)-en-28-oyl]-4-piperidine butyric acid (39): 20 mg (41% yield) starting from 50 mg of 38, white amorphous powder. Mp 116-118° C. MS (ESI+) m/z: 738.6 (M⁺+H), (ESI−) m/z: 736.5 (M⁻−H) for C₄₅H₇₁NO₇. ¹H NMR (300 MHz, CDCl₃): δ 4.72, 4.57 (1H each, s, H-29), 4.47 (1H, t, J=7.5, H-3), 3.65-3.50 (4H, m, 28-CON(CH₂CH₂)₂CH—), 2.98 (1H, m, H-19), 2.64-2.42 (2H, m, H-2′), 2.30 (2H, t, J=7.2 Hz, —CH₂COOH), 1.68 (3H, s, H-30), 1.27, 1.25 (3H each, s, 2×CH₃-3′), 0.95, 0.93, 0.84, 0.83, 0.82 (3H each, s, 5×CH₃). [α]²⁵_{D} −27.7° (c=0.30, MeOH).

N-[3β-O-(4′,4′-Dimethylglutaryl)-lup-20(29)-en-28-oyl]-4-piperidine butyric acid (40): 12 mg (38% yield) starting from 30 mg of 38, white amorphous powder. Mp 143-145° C. MS (ESI+) m/z: 752.4 (M⁺+H), (ESI−) m/z: 750.4 (M⁻−H) for C₄₆H₇₃NO₇. ¹H NMR (300 MHz, CDCl₃): δ 4.72, 4.57 (1H each, s, H-29), 4.47 (1H, t, J=7.5, H-3), 3.65-3.50 (4H, m, 28-CON(CH₂CH₂)₂CH—), 3.00 (1H, m, H-19), 2.35-2.30 (4H, m, H-2′, —CH₂COOH), 1.68 (3H, s, H-30), 1.27, 1.25 (3H each, s, 2×CH₃-3′), 0.96 (6H, s, 2×CH₃), 0.89, 0.86, 0.82 (3H each, s, 3×CH₃). [α]²⁵_{D} −23.1° (c=0.20, MeOH).

N′-[N-[3β-lup-20(29)-en-28-oyl]-4-piperidine-butanoyl]-aminomethane (45): 17 mg (48% yield) starting from 30 mg of 41, white amorphous powder. Mp 166-169° C. MS (ESI+) m/z: 751.6 (M⁺+H) for C₄₆H₇₄N₂O₆. ¹H NMR (300 MHz, CDCl₃): δ 5.43 (1H, br s, —CONHCH₃), 4.69, 4.54 (1H each, s, H-29), 4.47 (1H, t, J=7.5, H-3), 3.68-3.60 (4H, m, 28-CON(CH₂CH₂)₂CH—), 2.98 (1H, m, H-19), 2.79 (3H, d, J=3.4 Hz, —CONHCH₃), 2.67-2.62 (2H, m, H-2′), 2.14 (2H, t, J=6.8 Hz, —CH₂CONHCH₃), 1.65 (3H, s, H-30), 1.30, 1.25 (3H each, s, 2×CH₃-3′), 0.93, 0.91 (3H each, s, 2×CH₃), 0.90, 0.80, 0.79 (3H each, s, 3×CH₃). [α]²⁵_{D} −25.0° (c=0.12, MeOH).

N′-[N-[3β-O-(3′,3′-Dimethylsuccinyl)-lup-20(29)-en-28-oyl]-4-piperidine-butanoyl]-morpholine (46): 23 mg (55% yield) starting from 35 mg of 42, off-white amorphous powder. Mp 122-124° C. MS (ESI+) m/z: 807.6 (M⁺+H) for C₄₉H₇₈N₂O₇. ¹H NMR (300 MHz, CDCl₃): δ 4.69, 4.54 (1H each, s, H-29), 4.45 (1H, t, J=6.9, H-3), 3.66-3.61 (8H, m, 28-CON(CH₂CH₂)₂CH—, —CON(CH₂CH₂)₂O), 3.45-3.42 (4H, m, —CON(CH₂CH₂)₂O), 2.99-2.82 (1H, m, H-19), 2.67-2.52 (2H, m, H-2′), 2.28 (2H, t, J=7.8 Hz, —CH₂CON(CH₂CH₂)₂O), 1.65 (3H, s, H-30), 1.26 (6H, s, 2×CH₃-3′), 0.92, 0.90 (3H each, s, 2×CH₃), 0.79 (6H, s, 2×CH₃), 0.77 (3H, s, CH₃). [α]²⁵_{D} −19.1° (c=0.41, MeOH).

N′-[N-[3β-O-(3′,3′-Dimethylsuccinyl)-lup-20(29)-en-28-oyl]-4-piperidine-butanoyl]-2-aminoethylmorpholine (47): 14 mg (41% yield) starting from 30 mg of 43, white amorphous powder. Mp 121-123° C. MS (ESI+) m/z: 850.4 (M⁺+H) for C₅₁H₈₃N₃O₇. ¹H NMR (300 MHz, CDCl₃): δ 7.01 (1H, br, s, —CONHCH₂—), 4.70, 4.54 (1H each, s, H-29), 4.45 (1H, t, J=10.2, H-3), 3.81-3.78 (8H, m, 28-CON(CH₂CH₂)₂CH—, —CH₂N(CH₂CH₂)₂O), 3.48 (2H, m, —CONHCH₂—), 2.85-2.70 (7H, m, —CH₂N(CH₂CH₂)₂O, H-19), 2.60-2.52 (2H, m, H-2′), 2.14 (2H, t, J=7.5 Hz, —CH₂CONHCH₂—), 1.25 (6H, s, 2×CH₃-3′), 1.63 (3H, s, H-30), 0.93 (3H each, s, 2×CH₃), 0.90, 0.79, 0.72 (3H each, s, 3×CH₃). [α]²⁵_{D} −18.0° (c=0.16, MeOH).

N′-[N-[3β-O-(3′,3′-Dimethylsuccinyl)-lup-20(29)-en-28-oyl]-4-piperidine-butanoyl]-3-aminopropylmorpholine (48): 17 mg (47% yield) starting from 30 mg of 44, white amorphous powder. Mp 125-127° C. MS (ESI+) m/z: 864.6 (M⁺+H) for C₅₂H₈₅N₃O₇. ¹H NMR (300 MHz, CDCl₃): δ 6.76 (1H, br, s, —CONHCH₂—), 4.69, 4.54 (1H each, s, H-29), 4.30 (1H, m, H-3), 3.81-3.67 (8H, m, 28-CON(CH₂CH₂)₂CH—, —CH₂N(CH₂CH₂)₂O), 3.28 (2H, m, —CONHCH₂—), 2.96 (1H, m, H-19), 2.80-2.73 (6H, m, —CH₂N(CH₂CH₂)₂O), 2.60-2.52 (2H, m, H-2′), 2.16 (2H, t, J=7.5 Hz, —CH₂CONHCH₂—), 1.65 (3H, s, H-30), 1.25, 1.24 (6H, s, 2×CH₃-3′), 0.92, 0.90 (3H each, s, 2×CH₃), 0.80, 0.79, 0.78 (3H each, s, 3×CH₃). [α]²⁵_{D} −14.1° (c=0.24, MeOH).

In Vitro Metabolic Stability Assessment.

Materials. BA-derivatives 23 and 38 were synthesized and characterized in our study. NADPH, MgCl₂, KH₂PO₄, formic acid and ammonium acetate were purchased from Sigma-Aldrich. Reference compounds (fast-metabolized: buspirone, propranolol; moderate-metabolized: atenolol; and slow-metabolized: imipramine) were also purchased from Sigma-Aldrich. HPLC-grade acetonitrile and water was purchased from VWR. Pooled human liver microsomes (Lot No#70196) were purchased from BD biosciences (Woburn, Mass.).

Sample Preparation. Stock solutions of 23 and 38 (1 mg/mL) were prepared by dissolving the pure compound in methanol and stored at 4° C. For measurement of metabolic stability, four reference compounds as well as test compounds 23 and 38 were brought to a final concentration of 3 μM with 0.1 M potassium phosphate buffer at pH 7.4, which contained 0.2 mg/mL human liver microsome and 5 mM MgCl₂. The incubation volumes were 800 μL. Reactions were started by adding 80 μL of NADPH (final concentration of 1.0 mM) and stopped by taking the aliquots over time, then adding to 1.5 volumes of ice-cold acetonitrile. Incubations of all samples were conducted in duplicate. For each sample, 100 μL aliquots were taken out at 0, 5, 15, 30, 60, 120 min time points. After addition of 150 μL ice-cold acetonitrile, the mixture was centrifuged at 12,000 rpm for 5 min at 0° C. The supernatant was collected and 20 μL of the supernatant was directly injected to LCMS. The following controls were also conducted: 1) positive control incubations that contain liver microsomes, NADPH and the fast-metabolized substrate propranolol; 2) negative control incubations that omit NADPH; and 3) baseline control that only contain liver microsomes and NADPH.

HPLC-MS Conditions. Analysis was carried out on Shimadzu LCMS-20 with an electrospray ionization source (ESI). An Alltima C18 5μm 150 mm×2.1 mm column was used with a gradient elution at a flow rate of 1.5 mL/min. The initial elution condition was acetonitrile (B) in water (A, with 0.1% formic acid and 5 mM ammonium acetate) at 55%. After staying at initial condition for 3 min, the concentration of B increased linearly to 90% at 15 min, and stayed at 90% for 2 min. The mobile phase was then returned to the initial condition and re-equilibrated for 3 min. The MS conditions were optimized to detector voltage: +1.35 kV, acquisition mode: SIM of the appropriate molecular weights of the testing compounds. The CDL temperature is 200° C., heat block is 230° C. and neutralizing gas flow is 1.5 L/min. Samples were injected by auto-sampler. Electrospray ionization was operated in the positive ion mode. Full-scan spectra were also monitored over the range of 180-1000 m/z.

HIV-1_{IIIB} Replication Inhibition Assay in MT-2 Lymphocytes. The evaluation of HIV-1 inhibition was carried out as follows. The human T-cell line, MT-2, was maintained in continuous culture with complete medium (RPMI 1640 with 10% fetal calf serum supplemented with L-glutamine at 5% CO₂ and 37° C. Test samples were first dissolved in dimethyl sulfoxide (DMSO) at a concentration of 10 mg/mL to generate master stocks with dilutions made into tissue culture media to generate working stocks. The following drug concentrations were routinely used for screening: 100, 20, 4 and 0.8 μg/mL. For agents found to be active, additional dilutions were prepared for, subsequent testing so that an accurate EC₅₀ value could be determined. Test samples were prepared, and to each sample well, was added 90 μL of media containing MT-2 cells at 3×10⁵ cells/mL and 45 μL of virus inoculum (HIV-1_{IIIB} isolate) containing 125 TCID₅₀. Control wells containing virus and cells only (no drug) and cells only (no virus or drug) were also prepared. A second identical set of samples were added to cells under the same conditions without virus (mock infection) for cytotoxicity determinations (CC₅₀ defined below). In addition, AZT and bevirimat were also assayed during each experiment as positive drug controls. On day 4 PI, the assay was terminated and culture supernatants were harvested for p24 antigen ELISA analysis. The compound cytotoxicity was determined by XTT using the mock-infected sample wells. The detailed procedure was described previously.^{31,32} If a test sample inhibited virus replication and was not cytotoxic, its effects were reported in the following terms: EC₅₀, the concentration of the test sample that was able to suppress HIV replication by 50%; CC₅₀, the concentration of test sample that was toxic to 50% of the mock-infected cells; and therapeutic index (TI), the ratio of the CC₅₀ to EC₅₀.

HIV-1_{NL4-3} Replication Inhibition Assay in MT-4 Lymphocytes. A previously described HIV-1 infectivity assay was used.^{28,33} A 96-well microtiter plate was used to set up the HIV-1_{NL4-3} replication screening assay. NL4-3 variants at a multiplicity of infection (MOI) of 0.01 were used to infect MT4 cells. Culture supernatants were collected on day 4 post-infection for the p24 antigen capture using an ELISA kit from ZeptoMetrix Corporation (Buffalo, N.Y.).

### Example 2

The incorporation of long ethylene glycol side chains at the C-28 position of bevirimat was examined. Compounds 20 and 21 (Scheme B) were successfully prepared as described in Scheme 4. These two compounds displayed increased solubility and decreased protein binding affinity, while keeping high anti-maturation activity. The bioassay data for compounds 20 and 21 are summarized in Table 3.

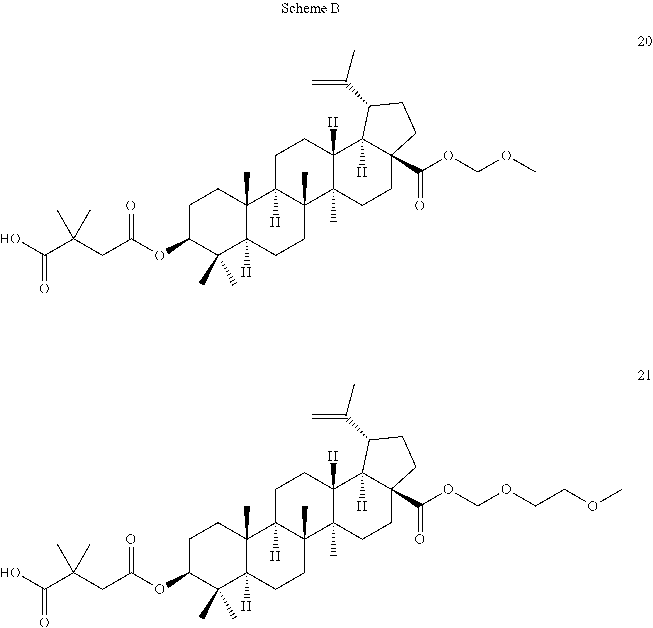

### 3. Bioassay Data for Compounds 20 and 21.

| Compounds | EC₅₀ (μM) | CC₅₀ (μM) | TI |
|---|---|---|---|
| bevirimat | 0.063 | 16.27 | 258.2 |
| 20 | 0.170 | 12.40 | 72.9 |
| 21 | 0.046 | 12.04 | 261.7 |

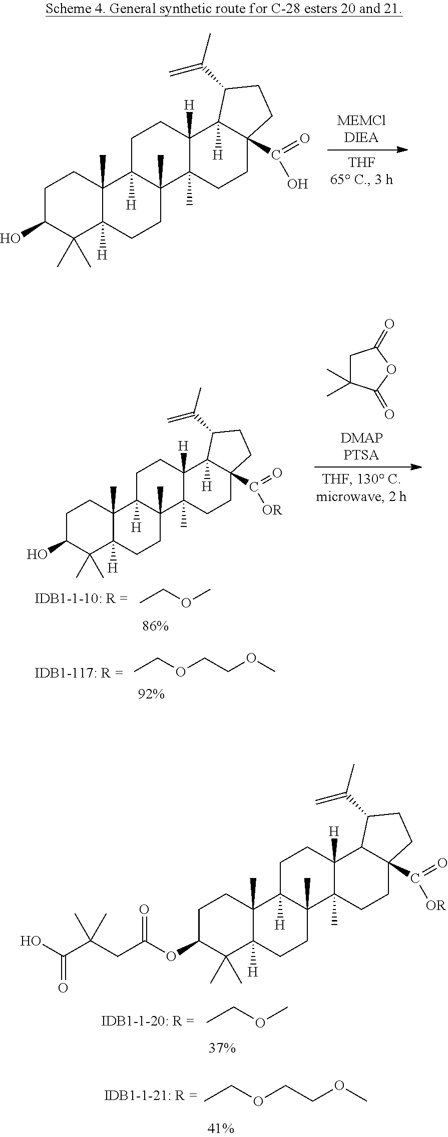

### REFERENCES

(1) UNAIDS. AIDS epidemic update, December 2007.
(2) Barbaro, G.; Scozzafava, A.; Mastrolorenzo, A.; Supuran, C. T. Highly active antiretroviral therapy: current state of the art, new agents and their pharmacological interactions useful for improving therapeutic outcome. Curr Pharm Des 2005, 11, 1805-1843.
(3) Ho, D. D.; Neumann, A. U.; Perelson, A. S.; Chen, W.; Leonard, J. M.; Markowitz, M. Rapid turnover of plasma virions and CD4 lymphocytes in HIV-1 infection. Nature 1995, 373, 123-126.
(4) Hammer, S. M.; Katzenstein, D. A.; Hughes, M. D.; Gundacker, H.; Schooley, R. T.; Haubrich, R. H.; Henry, W. K.; Lederman, M. M.; Phair, J. P.; Niu, M.; Hirsch, M. S.; Merigan, T. C. A trial comparing nucleoside monotherapy with combination therapy in HIV-infected adults with CD4 cell counts from 200 to 500 per cubic millimeter. AIDS Clinical Trials Group Study 175 Study Team. N Engl Med 1996, 335, 1081-1090.
(5) Gulick, R. M.; Meibohm, A.; Havlir, D.; Eron, J. J.; Mosley, A.; Chodakewitz, J. A.; Isaacs, R.; Gonzalez, C.; McMahon, D.; Richman, D. D.; Robertson, M.; Mellors, J. W. Six-year follow-up of HIV-1-infected adults in a clinical trial of antiretroviral therapy with indinavir, zidovudine, and lamivudine. Aids 2003, 17, 2345-2349.
(6) Chun, T. W.; Carruth, L.; Finzi, D.; Shen, X.; DiGiuseppe, J. A.; Taylor, H.; Hermankova, M.; Chadwick, K.; Margolick, J.; Quinn, T. C.; Kuo, Y. H.; Brookmeyer, R.; Zeiger, M. A.; Barditch-Crovo, P.; Siliciano, R. F. Quantification of latent tissue reservoirs and total body viral load in HIV-1 infection. Nature 1997, 387, 183-188.
(7) Finzi, D.; Hermankova, M.; Pierson, T.; Carruth, L. M.; Buck, C.; Chaisson, R. E.; Quinn, T. C.; Chadwick, K.; Margolick, J.; Brookmeyer, R.; Gallant, J.; Markowitz, M.; Ho, D. D.; Richman, D. D.; Siliciano, R. F. Identification of a reservoir for HIV-1 in patients on highly active antiretroviral therapy. Science 1997, 278, 1295-1300.
(8) Wong, J. K.; Hezareh, M.; Gunthard, H. F.; Havlir, D. V.; Ignacio, C. C.; Spina, C. A.; Richman, D. D. Recovery of replication-competent HIV despite prolonged suppression of plasma viremia. Science 1997, 278, 1291-1295.
(9) Piscitelli, S. C.; Flexner, C.; Minor, J. R.; Polis, M. A.; Masur, H. Drug interactions in patients infected with human immunodeficiency virus. Clin Infect Dis 1996, 23, 685-693.
(10) Louie, M.; Markowitz, M. Goals and milestones during treatment of HIV-1 infection with antiretroviral therapy: a pathogenesis-based perspective. Antiviral Res 2002, 55, 15-25.
(11) Boden, D.; Hurley, A.; Zhang, L.; Cao, Y.; Guo, Y.; Jones, E.; Tsay, J.; Ip, J.; Farthing, C.; Limoli, K.; Parkin, N.; Markowitz, M. HIV-1 drug resistance in newly infected individuals. Jama 1999, 282, 1135-1141.
(12) Wegner, S. A.; Brodine, S. K.; Mascola, J. R.; Tasker, S. A.; Shaffer, R. A.; Starkey, M. J.; Barile, A.; Martin, G. J.; Aronson, N.; Emmons, W. W.; Stephan, K.; Bloor, S.; Vingerhoets, J.; Hertogs, K.; Larder, B. Prevalence of genotypic and phenotypic resistance to anti-retroviral drugs in a cohort of therapy-naive HIV-1 infected US military personnel. Aids 2000, 14, 1009-1015.
(13) Hammer, S. M.; Pedneault, L. Antiretroviral resistance testing comes of age. Antivir Ther 2000, 5, 23-26.
(14) Kashiwada, Y.; Hashimoto, F.; Cosentino, L. M.; Chen, C. H.; Garrett, P. E.; Lee, K. H. Betulinic acid and dihydrobetulinic acid derivatives as potent anti-HIV agents. J Med Chem 1996, 39, 1016-1017.
(15) Li, F.; Goila-Gaur, R.; Salzwedel, K.; Kilgore, N. R.; Reddick, M.; Matallana, C.; Castillo, A.; Zoumplis, D.; Martin, D. E.; Orenstein, J. M.; Allaway, G. P.; Freed, E. O.; Wild, C. T. PA-457: a potent HIV inhibitor that disrupts core condensation by targeting a late step in Gag processing. Proc Natl Acad Sci USA 2003, 100, 13555-13560.
(16) Yu, D.; Wild, C. T.; Martin, D. E.; Morris-Natschke, S. L.; Chen, C. H.; Allaway, G. P.; Lee, K. H. The discovery of a class of novel HIV-1 maturation inhibitors and their potential in the therapy of HIV. Expert Opin Investig Drugs 2005, 14, 681-693.
(17) Martin, D. E.; Blum, R.; Wilton, J.; Doto, J.; Galbraith, H.; Burgess, G. L.; Smith, P. C.; Ballow, C. Safety and pharmacokinetics of Bevirimat (PA-457), a novel inhibitor of human immunodeficiency virus maturation, in healthy volunteers. Antimicrob Agents Chemother 2007, 51, 3063-3066.
(18) Smith, P. F.; Ogundele, A.; Forrest, A.; Wilton, J.; Salzwedel, K.; Doto, J.; Allaway, G. P.; Martin, D. E. Phase I and II study of the safety, virologic effect, and pharmacokinetics/pharmacodynamics of single-dose 3-o-(3′,3′-dimethylsuccinyl)betulinic acid (bevirimat) against human immunodeficiency virus infection. Antimicrob Agents Chemother 2007, 51, 3574-3581.
(19) Soler, F.; Poujade, C.; Evers, M.; Carry, J. C.; Henin, Y.; Bousseau, A.; Huet, T.; Pauwels, R.; De Clercq, E.; Mayaux, J. F.; Le Pecq, J. B.; Dereu, N. Betulinic acid derivatives: a new class of specific inhibitors of human immunodeficiency virus type 1 entry. J Med Chem 1996, 39, 1069-1083.
(20) Labrosse, B.; Treboute, C.; Alizon, M. Sensitivity to a nonpeptidic compound (RPR103611) blocking human immunodeficiency virus type 1 Env-mediated fusion depends on sequence and accessibility of the gp41 loop region. J Virol 2000, 74, 2142-2150.
(21) Sun, I. C.; Chen, C. H.; Kashiwada, Y.; Wu, J. H.; Wang, H. K.; Lee, K. H. Anti-AIDS agents 49. Synthesis, anti-HIV, and anti-fusion activities of IC9564 analogues based on betulinic acid. J Med Chem 2002, 45, 4271-4275.
(22) Qian, K., Morris-Natschke, S. L., Lee, K. H. HIV Entry Inhibitors and Their Potential in HIV Therapy. Medicinal Research Reviews 2009, 29, 369-393.
(23) Mayaux, J. F.; Bousseau, A.; Pauwels, R.; Huet, T.; Henin, Y.; Dereu, N.; Evers, M.; Soler, F.; Poujade, C.; De Clercq, E.; et al. Triterpene derivatives that block entry of human immunodeficiency virus type 1 into cells. Proc Natl Acad Sci USA 1994, 91, 3564-3568.
(24) Lai, W.; Huang, L.; Ho, P.; Li, Z.; Montefiori, D.; Chen, C. H. Betulinic Acid Derivatives That Target gp120 and Inhibit Multiple Genetic Subtypes of Human Immunodeficiency Virus Type 1. Antimicrob Agents Chemother 2008, 52, 128-136.
(25) Huang, L.; Zhang, L.; Chen, C. H. Potential drug targets on the HIV-1 envelope glycoproteins, gp120 and gp41. Curr Pharm Des 2003, 9, 1453-1462.
(26) Evers, M.; Poujade, C.; Soler, F.; Ribeill, Y.; James, C.; Lelievre, Y.; Gueguen, J. C.; Reisdorf, D.; Morize, I.; Pauwels, R.; De Clercq, E.; Henin, Y.; Bousseau, A.; Mayaux, J. F.; Le Pecq, J. B.; Dereu, N. Betulinic acid derivatives: a new class of human immunodeficiency virus type 1 specific inhibitors with a new mode of action. J Med Chem 1996, 39, 1056-1068.
(27) Wen, Z.; Martin, D. E.; Bullock, P.; Lee, K. H.; Smith, P. C. Glucuronidation of anti-HIV drug candidate bevirimat: identification of human UDP-glucuronosyltransferases and species differences. Drug Metab Dispos 2007, 35, 440-448.
(28) Huang, L.; Ho, P.; Lee, K. H.; Chen, C. H. Synthesis and anti-HIV activity of bi-functional betulinic acid derivatives. Bioorg Med Chem 2006, 14, 2279-2289.
(29) Sun, I. C.; Wang, H. K.; Kashiwada, Y.; Shen, J. K.; Cosentino, L. M.; Chen, C. H.; Yang, L. M.; Lee, K. H. Anti-AIDS agents. 34. Synthesis and structure-activity relationships of betulin derivatives as anti-HIV agents. J Med Chem 1998, 41, 4648-4657.
(30) Leeds, J. P., Kirst, H. A. A Mild Single-step Reduction of Oximes to Amines. Synth. Commun 1988, 18, 777-782.
(31) Qian, K.; Nakagawa-Goto, K.; Yu, D.; Morris-Natschke, S. L.; Nitz, T. J.; Kilgore, N.; Allaway, G. P.; Lee, K. H. Anti-AIDS agents 73: structure-activity relationship study and asymmetric synthesis of 3-O-monomethylsuccinyl-betulinic acid derivatives. Bioorg Med Chem Lett 2007, 17, 6553-6557.
(32) Roehm, N. W.; Rodgers, G. H.; Hatfield, S. M.; Glasebrook, A. L. An improved colorimetric assay for cell proliferation and viability utilizing the tetrazolium salt XTT. J Immunol Methods 1991, 142, 257-265.
(33) Yu, D.; Sakurai, Y.; Chen, C. H.; Chang, F. R.; Huang, L.; Kashiwada, Y.; Lee, K. H. Anti-AIDS agents 69. Moronic acid and other triterpene derivatives as novel potent anti-HIV agents. J Med Chem 2006, 49, 5462-5469.

1. A compound according to Formula (I): 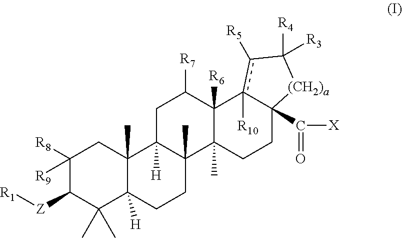
wherein:
a is 1 or 2;
Z is O, S, NH, or N-alkyl;
R₁ is a hydrogen, acyl carboxylic acid, C₂ to C₂₀ substituted or unsubstituted carboxyacyl, or a substituent of the formula: 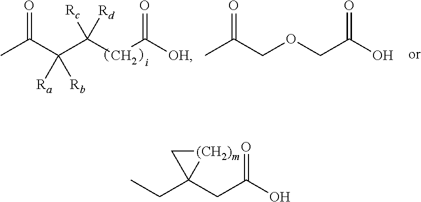
wherein Rₐ, R_{b}, R_{c} and R_{d} are the same or different and are each independently selected from the group consisting of hydrogen and lower alkyl, i is an integer from 0 to 3, and m is an integer from 1 to 4;
X is polyalkylene oxide, heteroalkylene, or —NR₂ₐR_{2b}, wherein R₂ₐ is H, loweralkyl, heteroalkylene, or polyalkylene oxide and R_{2b} is H, heteroalkylene, polyalkylene oxide, or a substituent of the formula: 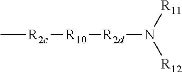
where R_{2c} is C2 to C10 saturated or unsaturated alkylene, R_{2d} is present or absent and when present is C1 to C5 saturated or unsaturated alkylene, R₁₀ is CONH, NHCO, NH, SH, or O, and R₁₁ and R₁₂ are each H, loweralkyl, heteroalkyl, carboxy, amino acid, or a peptide, or R₁₁ and R₁₂ together form with the N to which they are joined cycloalkyl or heterocycloalkyl;
or R₂ₐ and R_{2b} together are C3 to C5 alkylene, which alkylene is substituted or unsubstituted;
R₃ and R₄ are either H or lower alkyl (e.g., methyl);
R₅ is H, lower alkyl, or —CRᵢRᵢᵢRᵢᵢᵢ, where:
Rᵢ is a methyl radical or forms with Rᵢᵢ a methylene radical or an oxo radical;
Rᵢᵢ is a hydrogen atom or forms with Rᵢ or Rᵢᵢᵢ a methylene radical or an oxo radical; and
Rᵢᵢᵢ is a hydroxyl, methyl, hydroxymethyl, —CH₂OR′ᵢᵢᵢ, —CH₂SR′ᵢᵢᵢ, or —CH₂NHR′ᵢᵢᵢ, which R′ᵢᵢᵢ is alkyl, hydroxyalkyl, dihydroxyalkyl, acetamidoalkyl, acetyl, heteroalkylene, or polyalkylene oxide; or Rᵢᵢᵢ is an amino radical substituted with hydroxyalkyl, carboxyhydroxyalkyl, or dialkylamino, the alkyl parts of which can form, with the nitrogen atom to which they are joined, a 5- or 6-membered heterocycle optionally containing 1 or 2 additional hetero atoms selected from the group consisting of O, S, NH, and N-alkyl;
or R₅ is a bond to an immediately adjacent carbon atom (thus forming a double bond in the ring between immediately adjacent carbon atoms);
R₆ and R₇ are either H or form a bond with one another (thus forming a double bond between their immediately adjacent carbon atoms);
R₈ and R₉ are either hydrogen or together form an oxo radical;
R₁₀ is either H or a bond with an immediately adjacent carbon atom (thus forming a double bond in the ring between immediately adjacent carbon atoms); and
the dashed line in Formula (I) is an optional double bond;
or a stereoisomer, enantiomer, tautomer thereof or mixtures thereof;
or a pharmaceutically acceptable salt or prodrug thereof.

2. The compound of claim 1, wherein X is —NR₂ₐR_{2b}.

3. The compound of claim 2, wherein R_{2b} is a substituent of the formula: 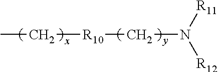
where x is an integer from 2 to 10, y is an integer from 0 to 5.

4. The compound of claim 2, wherein R₂ₐ and R_{2b} together form a substituent of the formula: 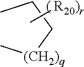
wherein:
q is 1, 2, or 3;
r is 1, 2 or 3; and
each R₂₀ is independently selected from the group consisting of alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, aryloxy, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxyacylamino, and aminoacyloxy.

5. The compound of claim 4, wherein R₂₀ is a substituent of the formula: 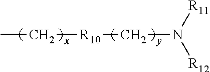
where x is an integer from 1 to 10, y is an integer from 1 to 5, R₁₀ is CONH, NHCO, NH, SH, or O, and R₁₁ and R₁₂ are each H, loweralkyl, heteroalkyl, carboxy, amino acid, or a peptide, or R₁₁ and R₁₂ together form with the N to which they are joined cycloalkyl or heterocycloalkyl.

6. The compound of claim 1, wherein R₁ is a hydrogen, or a substituent of the formula: 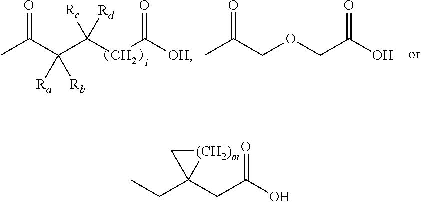
wherein Rₐ, R_{b}, R_{c} and R_{d} are the same or different and are each independently selected from the group consisting of hydrogen or lower alkyl, i is an integer from 0 to 3, and m is an integer from 1 to 4.

7. The compound of claim 1, wherein R₁ is 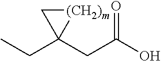
and m is an integer from 1 to 4.

8. The compound of claim 1, wherein R₁ is C₂ to C₂₀ substituted or unsubstituted carboxyacyl.

9. The compound of claim 1, wherein R₁ contains at least one asymmetric center with a (S) configuration.

10. The compound of claim 1, wherein R₆ and R₁₀ are each H.

11. The compound of claim 1, wherein R₈ and R₉ are each H.

12. The compound of claim 1, wherein R₅ is —CRᵢRᵢᵢRᵢᵢᵢ.

13. The compound of claim 12, wherein Rᵢ and Rᵢᵢᵢ together form a methylene radical.

14. The compound of claim 12, wherein Rᵢᵢ is methyl.

15. The compound of claim 1, wherein R₃ and R₄ are each H.

16. The compound of claim 1, wherein R₆ and R₇ are each H.

17. The compound of claim 6, wherein X is heteroalkylene or polyalkylene oxide.

18. The compound of claim 17, wherein X is heteroalkylene, Z is O, and R1 is C₂ to C₂₀ substituted or unsubstituted carboxyacyl.

19. The compound of claim 18, wherein R₁ contains at least one asymmetric center with a (S) configuration.

20. A compound according to claim 1, wherein said compound has the structure:

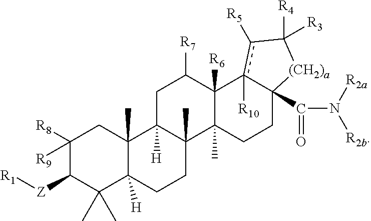

21. The compound of claim 20, wherein R₂ₐ is heteroalkylene or polyalkylene oxide.

22. A composition comprising a compound of claim 1 in a pharmaceutically acceptable carrier.

23. The composition of claim 22, further comprising at least one additional antiviral agent.

24. A method of treating a retroviral infection in a subject in need thereof, comprising administering said subject a compound of claim 1 in a treatment-effective amount.

25. The method of claim 24, wherein said retroviral infection is an HIV-1 infection.

26. The method of claim 24, further comprising concurrently administering said subject at least one additional antiviral agent in a treatment-effective amount.

27-28. (canceled)
